(19) United States
(12) Patent Application Publication
(10) Pub. No.: US 2025/0082716 A1
(43) Pub. Date: Mar. 13, 2025

(54) REPRESSED ANG 1-7 IN COVID-19 IS INVERSELY ASSOCIATED WITH INFLAMMATION AND COAGULATION

(71) Applicant: University of Virginia Patent Foundation, Charlottesville, VA (US)

(72) Inventors: William A. Petri, JR., Charlottesville, VA (US); Rebecca Marie Carpenter, Charlottesville, VA (US); Rachel Letteri, Charlottesville, VA (US); Sanford H Feldman, Ruckersville, VA (US); Nicholas R. Natale, Charlottesville, VA (US)

(73) Assignee: University of Virginia Patent Foundation, Charlottesville, VA (US)

(21) Appl. No.: 18/560,901

(22) PCT Filed: May 16, 2022
(86) PCT No.: PCT/US2022/029465

§ 371 Date: Nov. 14, 2023

Related U.S. Application Data
(60) Provisional application No. 63/188,927, filed on May 14, 2021.

(51) Int. Cl. A61K 38/08; A61K 31/4178; A61K 45/06

(52) U.S. Cl. CPC A61K 38/085; A61K 31/4178; A61K 45/06

(57) ABSTRACT

Provided are methods for treating subjects with coronavirus infections. The methods include providing a subject infected with a coronavirus resulting in a prothrombotic condition in addition to being infected by a coronavirus, and administering to the subject an angiotensin (1-7) peptide or an analog or derivative thereof, a Mas Receptor (MasR) agonist, or any combination thereof. The subject may be suffering from COVID-19 disease, including but not limited to a thrombotic complication, an adverse pregnancy outcome, and/or a complication resulting from an underlying prothrombotic state. Also provided are compositions that include Ang (1-7) peptides, analogs, and/or derivatives thereof that are associated with degradable and/or non-degradable polymers having electrostatic interactions therewith, hydrophobic interaction therewith, hydrogen bonding interactions therewith, or any combination thereof. Also provided are uses of Ang (1-7) peptides, analogs, and/or derivatives thereof and/or a Mas Receptor (MasR) agonists for treating subjects infected with coronaviruses and/or for preparing medicaments therefore.

### CROSS REFERENCE TO RELATED APPLICATION

The presently disclosed subject matter claims the benefit of U.S. Provisional Patent Application Ser. No. 63/188,927, filed May 14, 2021, the disclosure of which incorporated herein by reference in its entirety.

### REFERENCE TO SEQUENCE LISTING SUBMITTED ELECTRONICALLY

The content of the electronically submitted sequence listing in ASCII text file (Name: 3062_157_PCT_ST25.txt; Size: 107 kilobytes; and Date of Creation: May 16, 2022) filed with the instant application is incorporated herein by reference in its entirety.

### GOVERNMENT INTEREST

This invention was made with government support under Grant No. AI124214 awarded by The National Institutes of Health. The government has certain rights in the invention.

### TECHNICAL FIELD

The presently disclosed subject matter relates to compositions and methods for preventing and/or treating diseases, disorders, and/or conditions associated with coronavirus infections, which in some embodiments include use of angiotensin 1-7 peptides, mimetics thereof, analogs thereof, and/or derivatives thereof.

### BACKGROUND

Since its discovery in December 2019, SARS-CoV-2 has caused over one hundred million cases of COVID-19 resulting in more than 2 million deaths worldwide (World Health Organization, 2021). Symptoms of COVID-19 are highly variable and include but are not limited to fever, cough, shortness of breath, fatigue, new loss of taste or smell, and diarrhea (Behzad et al., 2020). Clinical responses range from minor symptoms to hyperimmune activation, hypercoagulopathy, multiorgan dysfunction and respiratory failure leading to prolonged ICU admissions and death (Behzad et al., 2020; Iba et al., 2020). Two years into the pandemic, emerging SARS-CoV-2 strains have led to increased worldwide transmission even among vaccinated populations highlighting the urgent need for further characterization of the pathophysiology of this disease and the continued development of effective therapeutics (World Health Organization, 2021).

The SARS-CoV-2 virus infects hosts by gaining entry to cells via its receptor angiotensin converting enzyme 2 (ACE2) which belongs to the renin-angiotensin system (RAS), a cascade of biologically active peptides, enzymes, and receptors central to fluid and electrolyte balance and the regulation of blood pressure (Wiese et al., 2020). Over the past two decades. ACE2 and its heptapeptide product angiotensin Ang 1-7 have increasingly been recognized as counterregulatory modulators of the classical RAS via activation of the Mas receptor (MasR; Gheblawi et al., 2020). ACE2 regulates the RAS by converting Ang I to Ang 1-9, and by cleaving a single amino acid from Ang II to form Ang 1-7 which is 500-fold more catalytically active than any other pathway leading to the formation of Ang 1-7 (Ferrario, 2011). Ang 1-7 then exerts its biologic effects by activating the MasR (see FIG. 1; see also Ponti et al., 2020). Ang II acts via the Ang II type-1 receptor (AT₁R) to induce vasoconstriction, inflammatory cytokine production and extracellular matrix synthesis (Pucci et al., 2021). Ang II also stimulates adrenal aldosterone production leading to sodium and fluid retention and an increase in blood pressure (Rieder et al., 2021). In contrast, Ang 1-7 via the MasR induces vasodilation and inhibits the production of proinflammatory cytokines along with other effects opposing those of Ang II (see FIG. 1; see also Rieder et al., 2021).

RAS dysregulation has been hypothesized as having a central role in the pathogenesis of severe coronaviral infections as early as 2003 after the first SARS-CoV outbreak (Pucci et al., 2021). After identification of ACE2 as the viral spike protein binding site, evidence emerged that ACE2 downregulation provides a molecular explanation for the observed severe respiratory failure caused by this virus (Kuba et al., 2005). Multiple studies in animals and humans have shown that attachment of the viral spike protein to the ACE2 receptor at least transiently reduces ACE2 expression by several mechanisms: induced cleavage and release of the soluble form of ACE2 by ADAM-17, virus-receptor complex internalization and receptor downregulation as a host-defense mechanism (Kuba et al., 2005; Chen et al., 2020; Henry et al., 2020). In vitro and in vivo animal studies further established evidence that ACE2 is an important modulator of acute lung injury, including the most severe form, acute respiratory distress syndrome (ARDS; see e.g., Kuba et al., 2005; Wösten-van Asperen et al., 2011; Uhal et al., 2012; Wösten-van Asperen et al., 2013; Gheblawi et al., 2020). Indeed, administration of the SARS-CoV spike protein to an acute lung injury mouse model reduces ACE2 expression and worsens the severity of injury (Kuba et al., 2005). ACE2 knockout mice are shown to have worsened oxygenation, increased inflammation, and lung edema in ARDS induced by acid aspiration or sepsis (Imai et al., 2005). In terms of disease pathogenesis. ACE2 is thought to have a protective role in lung injury and to act in opposition to ACE by downregulating and thereby mitigating the pro-inflammatory effects of Ang II and promoting the effects of Ang 1-7 via the Mas receptor (see FIG. 1; see also Imai et al., 2005; Wösten-van Asperen et al., 2011; Khan et al., 2017; Angell et al., 2021). In support of this hypothesis, the administration of recombinant ACE2 has been shown to reduce Ang II, increase Ang 1-7 and attenuate lung injury in acid-treated mice (Imai et al., 2005). These data support a robust hypothesis that COVID-19 is an acquired molecular disease characterized by RAS dysregulation, namely ACE2 downregulation, which leads to an exacerbated immune response, microcirculatory thrombosis, and fibrosis (Angeli et al., 2021; Ramos et al., 2021).

Observations from animal models have suggested RAS modulation as a potential therapeutic mechanism to treat COVID-19 and indeed clinical trials looking at the impact of Ang1-7 and human recombinant ACE2 are ongoing. However, evidence from human data, has shown that perhaps these hypotheses have been too optimistic (Pucci et al., 2021). Early studies have shown conflicting results with the majority of evidence pointing towards overactivation of the ACE2/Ang 1-7/MasR pathway and an overall increase in Ang 1-7 in COVID-19 (Pucci et al., 2021). Furthermore, little to no data in human studies supports the role of RAS dysregulation leading to the hypothesized downstream inflammatory and thrombotic overactivation in COVID-19. Thus, there is need for further investigation to determine if RAS dysfunction is responsible for at least some of the unique features of COVID-19 and to support the development of targeted therapeutics. Here we measured Ang II and Ang 1-7 as a proxy for ACE2 activity in plasma of COVID-19 patients to determine their relationship to severity of illness and markers of inflammation and coagulopathy.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter relates in some embodiments to methods for treating subjects with a coronavirus infection. In some embodiments, the methods comprise, consist essentially of, or consist of providing a subject infected with a coronavirus resulting in and/or having a prothrombotic condition in addition to being infected by a coronavirus; and administering to the subject an angiotensin (1-7) peptide or an analog or derivative thereof, a Mas Receptor (MasR) agonist, or any combination thereof. In some embodiments, the coronavirus is a SARS-CoV-1, MERS, or SARS-CoV-2 coronavirus. In some embodiments, the subject is suffering from COVID-19 disease. In some embodiments, the subject has and/or is at risk for developing a thrombotic complication, optionally a thrombotic complication selected from the group consisting of acute limb ischemia, abdominal and/or thoracic aortic thrombosis, mesenteric ischemia, myocardial infarction, venous thromboembolism, pulmonary embolism, cerebrovascular accident, and any form of systemic arterial embolism. In some embodiments, the subject is at an elevated risk of an adverse pregnancy outcome, optionally wherein the adverse pregnancy outcome is selected from the group consisting of intrauterine fetal death, stillbirth, fetal growth restriction, and preterm birth secondary to placental insufficiency, and further wherein the adverse pregnancy outcome results from and/or is secondary to thrombosis formation in the subject associated with the coronavirus infection. In some embodiments, the subject is at risk of a complication resulting from the coronavirus infection due to an underlying prothrombotic state, optionally wherein the underlying prothrombotic state results from pregnancy, malignancy, a genetic condition, and/or a rheumatologic condition that predisposes the subject to thrombosis formation.

In some embodiments of the presently disclosed methods, the administration is parenteral, rectal, oral, or a combination thereof. In some embodiments, the parenteral administration is intravenous, subcutaneous, inhalation, intradermal, transdermal, and/or transmucosal administration.

In some embodiments of the presently disclosed methods, the angiotensin (1-7) peptide comprises an amino acid sequence selected from the group consisting of Asp-Arg-Val-Tyr-Ile-His-Pro (SEQ ID NO: 1) or a functional equivalent thereof, Asp-Arg-Val-Ser-Ile-His-Cys (SEQ ID NO: 2) or a functional equivalent thereof, and Ala-Arg-Val-Ser-Ile-His-Cys (SEQ ID NO: 3) or a functional equivalent thereof.

In some embodiments of the presently disclosed methods, the angiotensin (1-7) peptide or the analog or derivative thereof is provided in composition comprising a degradable polymer having an electrostatic interaction with the angiotensin (1-7) peptide or the analog or derivative thereof, a non degradable polymer having an electrostatic interaction with the angiotensin (1-7) peptide or the analog or derivative thereof, a non-degradable polymer having a hydrophobic interaction with the angiotensin (1-7) peptide or the analog or derivative thereof, a non-degradable polymer having a hydrogen bonding interaction with the angiotensin (1-7) peptide or the analog or derivative thereof, or any combination thereof.

In some embodiments of the presently disclosed methods, the Mas Receptor (MasR) agonist is N-(Ethylcarbamoyl)-3-(4-((5-formyl-4-methoxy-2-phenyl-1H-imidazol-1-YL)methyl)phenyl)-5-isobutylthiophene-2-sulfonamide (AVEO991), an analog thereof, a derivative thereof, or any combination thereof.

In some embodiments of the presently disclosed methods, the subject is suffering from weight loss and/or olfactory nerve invasion by the coronavirus.

In some embodiments, the presently disclosed methods further comprise, consist essentially of, or consist of administering to the subject an ACE inhibitor, an Angiotensin II Receptor Blocker (ARB), or any combination thereof. In some embodiments, the ACE inhibitor is selected from the group consisting of benazepril, captopril, enalapril, fosinopril, lisinopril, moexioril, perindopril, quinapril, ramipril, and trandolapril; and/or the ARB is selected from the group consisting of candesartan, irbesartan, olmesartan, losartan, valsartan, telmisartan, and eprosartan; or any combination thereof.

In some embodiments, the presently disclosed subject matter also relates to compositions comprising, consisting essentially of, or consisting of a degradable polymer having an electrostatic interaction with the angiotensin (1-7) peptide or an analog or derivative thereof, a non-degradable polymer having an electrostatic interaction with an angiotensin (1-7) peptide or the analog or derivative thereof, a non-degradable polymer having a hydrophobic interaction with an angiotensin (1-7) peptide or the analog or derivative thereof, a non-degradable polymer having a hydrogen bonding interaction with and angiotensin (1-7) peptide or the analog or derivative thereof, or any combination thereof. In some embodiments, the composition is formulated for the treatment of a subject infected with a SEE coronavirus and having a prothrombic condition in addition to being infected by a coronavirus. In some embodiments, a composition of the presently disclosed subject matter is administered to a subject suffering from weight loss and/or olfactory nerve invasion by the coronavirus.

In some embodiments, the presently disclosed subject matter also relates to uses of angiotensin (1-7) peptides and/or analogs or derivatives thereof and/or Mas Receptor (MasR) agonists for the preparation for medicament for treating subjects infected with a coronavirus. In some embodiments, the subject is a subject infected with a coronavirus and having a prothrombic condition in addition to being infected by a coronavirus.

In some embodiments, the presently disclosed subject matter also relates to uses of angiotensin (1-7) peptides and/or analogs or derivatives thereof and/or Mas Receptor (MasR) agonists for treating subject infected with a coronavirus. In some embodiments, the subject is a subject infected with a coronavirus and having a prothrombic condition in addition to being infected by a coronavirus.

In some embodiments of the presently disclosed uses, the angiotensin (1-7) peptide and/or the analog or derivative thereof is provided in a composition comprising a degradable polymer having an electrostatic interaction with the angiotensin (1-7) peptide and/or the analog or derivative thereof, a non-degradable polymer having an electrostatic interaction with the angiotensin (1-7) peptide and/or the analog or derivative thereof, a non-degradable polymer having a hydrophobic interaction with the angiotensin (1-7) peptide and/or the analog or derivative thereof, a non-degradable polymer having a hydrogen bonding interaction with the angiotensin (1-7) peptide and/or the analog or derivative thereof, or any combination thereof.

In some embodiments of the presently disclosed uses, the subject is suffering from weight loss and/or olfactory nerve invasion by the coronavirus.

Accordingly, it is an object of the presently disclosed subject matter to provide compositions and methods for treating and/or preventing coronavirus infections, including but not limited to subjects who have prothrombotic conditions in addition to and/or as a consequence of being infected by a coronavirus.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following Description, Figures, and EXAMPLES.

### BRIEF DESCRIPTION OF THE FIGURES

FIG. 1. Schematic representation of the systemic renin-angiotensin system. Renin converts angiotensinogen into angiotensin I (Ang I). Angiotensin-converting enzyme (ACE) converts Ang I into angiotensin II (Ang II), which binds to Ang II type I receptor (AT₁R) through which it exerts its harmful inflammatory effects. Angiotensin-converting enzyme 2 (ACE2) converts the majority of Ang II to Ang 1-7, which activates the Mas receptor (MasR) signaling pathway with protective downstream effects on the microcirculatory environment. Attachment of SARS-CoV-2 spike protein to ACE2 induces cleavage and release of the soluble form of ACE2 by ADAM-17, virus-receptor complex internalization and receptor downregulation. Reduced ACE2 expression is hypothesized to cause an imbalance between the classical ACE/Ang 1/AT₁R and the protective ACE2/Ang 1-7/MasR aces that is central to the pathophysiology of coronavirus disease 2019 (COVID-19).

FIGS. 2A-2C. Reduced ACE2 activity evidenced by repressed Ang 1-7 and increased Ang II:Ang 1-7 ratios. (FIGS. 2A and 2B) Boxplots show the distribution of Ang 1-7 and Ang II stratified by COVID-19 status and adverse outcomes including hospitalization, need for oxygen supplementation, ventilation, and death. Above each outcome is the unadjusted p-value from non-parametric Wilcoxon Mann-Whitney tests. The number of unique patient samples quantified is as follows, Angiotensin II (n=202). Angiotensin 1-7 (n=229). (FIG. 2C) The log-transformed ratio of Ang II to Ang 1-7 is displayed. T-test p-values are included above each boxplot from analyzing each outcome category separately. Symbols are: * p<0.05, ** p<0.005, *** p<0.0005

FIGS. 3A-3D. Inverse relationship of Ang II and Ang 1-7 to mean arterial blood pressures (MAP). (FIGS. 3A and 3B) Linear regression between Ang II and Ang 1-7 levels and MAP averaged from all available readings taken on the day of sample collection. (FIGS. 3C and 3D) Linear regression between Ang II and Ang 1-7 and potassium levels collected from the day of sample collection. The number of measurements available on the day of sample collection are as follows, MAPS (n=156), Potassium (n=98).

FIG. 4. Correlations between angiotensin peptides, D-dimer, and pro-inflammatory cytokines in COVID-19. The correlation matrix depicts the Spearman correlation coefficient between Angiotensin 1-7, Angiotensin II D-dimer, and cytokine levels on a colorimetric scale from negative correlation in red (when shown in color; also, the scale at the right of the figure presents yellow to shades of orange to red at the bottom right, below 0, when shown in color) to positive correlation in blue (when shown in color; the scale at the top right of the figures presents light blue to dark blue, going up from zero, when shown in color). The cytokines displayed were selected based on significant associations with Angiotensin 1-7 levels using Kruskal-Wallis tests and analyzing each cytokine separately. The number of unique patient samples quantified is as follows, all cytokines (n=164), Angiotensin II (n=202), Angiotensin 1-7 (n=229), D-dimer retrospectively pulled from clinical records (n=54), Mean Arterial Pressure (MAP) (n=156).

FIGS. 5A and 5B. Low Ang 1-7 quartiles associated with high proportion of patients requiring hospitalization and oxygen supplementation. A higher proportion of patients with Angiotensin levels <66 μg/mL required hospitalization (FIG. 5A) and oxygen supplementation (FIG. 5B) when compared to patients with an Angiotensin level quantified at >187 μg/mL. * P-values were obtained from logistic regression. A value in the highest Ang 1-7 quartile (>187 μg/mL) was associated with a 95% reduction in odds of hospitalization (OR 0.05, CI 0.002 to 0.29, p=0.00573) and a 65% reduction in odds of requiring oxygen supplementation (OR 0.35, CI 0.12 to 0.94, p=0.0440) when compared to the reference lowest Ang 1-7 quartile (<66 μg/mL).

FIG. 6. Cytokine and D-dimer associations with Ang 1-7 quartiles. Associations between individual inflammatory/coagulation markers and Ang 1-7 quartiles (1st quartile=<66 μg/mL; 2nd quartile=66-103 pg/mL; 3rd quartile=103-187 pg/mL; and 4th quartile=>187 μg/mL) was detected using Kruskal-Wallis tests (p value<0.05 was considered statistically significant). Cytokines tested included, from left to right by row starting with the top row, soluble cluster of differentiation 40 (CD40) ligand (SCD40L), epidermal growth factor (EGF), Eotaxin, and fibroblast growth factor 2 (FGF-2); Fms-related tyrosine kinase 3 ligand (FLT-3L), Fractalkine/CX3CL 1, granulocyte-colony stimulating factor (G-CSF), and granulocyte/macrophage-colony stimulating factor (GM-CSF); GRO alpha/CXCL1 (GROa), Interferon Alpha 2 (IFNa2), Interferon Gamma (IFNγ), and Interleukin 1 alpha (IL-1a); Interleukin 1 beta (IL-1b), interleukin-1 receptor antagonist (IL-1RA), Interleukin 2 (IL-2), and Interleukin 3 (IL-3); Interleukin 4 (IL-4). Interleukin 5 (IL-5). Interleukin 6 (IL-6), and Interleukin 7 (IL-7); Interleukin 8 (IL-8), Interleukin 9 (IL-9). Interleukin 10 (IL-10), and Interleukin 12 p40 (IL-12p40); Interleukin 12 p70 (IL-12p70), Interleukin 13 (IL-13), Interleukin 15 (IL-15), and Interleukin 17 alpha (IL-17a); Interleukin 17E/Interleukin 25 (IL-17E/IL25), Interleukin 17F (IL-17F), Interleukin 18 (IL-18), and Interleukin 22 (IL-22); Interleukin 27 (IL-27), interferon γ-induced protein 10 kDa (IP-10), monocyte chemoattractant protein-1 (MCP-1), and monocyte chemoattractant protein-3 (MCP-3); macrophage colony-stimulating factor (M-CSF), macrophage-derived chemokine (MDC), monokine induced by gamma interferon (MIG), and macrophage inflammatory protein-1 alpha (MIP-1a); macrophage inflammatory protein-1 beta (MIP-1b), platelet-derived growth factor isoform AA (PDGF-AA), platelet-derived growth factor isofonn AA/BB (PDGF-AA/BB), and transforming growth factor alpha (TGfa); and tumor necrosis factor alpha (TNFa), tumor necrosis factor beta (TNFb), vascular endothelial growth factor (VEGF), and D-dimer. Each box includes data for the 1st quartile, 2nd quartile, 3rd quartile, and 4th quartile from left to right. The number of unique patient samples included was 164 for all cytokines and 54 for D-dimer retrospectively pulled from clinical records. ns: not significant. * p<0.05. ** p<0.01. *** p<0.001.

FIG. 7. Reactive increase in IL-10 correlated with proinflammatory cytokines. Proinflammatory cytokines. TNFa (top left), MCP-1 (bottom left), IL-6 (top right), and IL-1B (bottom right) along with IL-10 were log-transformed and then inputted into linear regression.

FIG. 8. daily body weights expressed as the percentage of starting body weight of hamsters inoculated intra-tracheally with 500,000 PFU SARS-CoV-2 South African variant that received various treatments. The day after inoculation, 90-100 gm LVG Golden Syrian male hamsters (Charles River Laboratories, Wilmington, Massachusetts, United States of America) were divided into four treatment groups: control subcutaneous pump implant with 10% DMSO in saline (virus only; n=3); pump implant delivering angiotensin 1-7 at 12 mg/kg/day (Ang 1-7; n=3); pump implant delivering angiotensin 1-7 at 12 mg/kg/day and the ACE inhibitor lisinopril ((2S)-1-[(2S)-6-amino-2-[[(1S)-1-carboxy-3-phenylpropyl]amino]hexanoyl]pyrrolidine-2-carboxylic acid) in drinking water at 50 mg/kg/day (Virus/Ang 1-7/lisinopril; n=4); and pump implant delivering the MasR agonist AVE0991 (N-(Ethylcarbamoyl)-3-(4-((5-formyl-4-methoxy-2-phenyl-1H-imidazol-1-YL)methyl)phenyl)-5-isobutylthiophene-2-sulfonamide) in 10% DMSO in propylene glycol at 0.6 mg/kg/day (AVE0991; n=4). Hamsters in the control group, and those receiving angiotensin 1-7 and lisinopril, lost weight and did not return to their pre-infection weights. Hamsters treated with angiotensin 1-7 or AVE0991 continued to gain weight through the entire course of infection.

FIGS. 9A and 9B. Implant delivery of angiotensin 1-7 agonist prevents SARS-CoV-2 infection of the hamster olfactory bulb. Olfactory bulbs were extracted on day-post-infection 7 from hamsters belonging to the treatment groups described in FIG. 8. RNA was isolated using Qiagen Viral RNA isolation kit and amplified using 2019-nCoV N1-P and 2019-nCoV N1-R primers with low mismatch target frequency (Vogels et al., 2020). A TaqMan probe was added to the amplicon to increase the sensitivity and specificity of the assay (2019-nCoV N3-P 5′-FAM-AYCACATTGGCACCCGCAATCCTG-BHQ 1; SEQ ID NO: 4). To amplify the copy number, the amplicon was cloned into pCR4 (Invitrogen Corp., Carlsbad, California, United States of America). The plasmid was then isolated with a plasmid miniprep kit (QIAGEN Sciences Inc. Germantown, Maryland, United States of America) and cut with a Bgl-1 restriction endonuclease. The Bgl-1 restriction endonuclease was heat inactivated. The plasmid insert concentration was quantified with a nanodrop spectrophotometer which was converted to copy number with a 660 gm/basepair mole+Avagadro's number conversion. A standard curve was generated through serial dilutions of the cut plasmid with insert to obtain copy number per ng total RNA isolated (FIGS. 9A and 9B). Treatment groups were as follows: A—control; B—Virus/Ang 1-7/lisinopril; C—Ang 1-7; and D—AVE0991.

FIG. 10: schematic showing exemplary degradable polymers for electrostatic encapsulation and controlled release of Ang 1-7. An exemplary degradable anionic polymer can be employed to encapsulate an exemplary cationic Ang 1-7 peptide, optionally a modified cationic model peptide, (Glycine-Arginine)₁₀, were produced at 1 mg/ml in 50 mM carbonate buffer (pH 10), and over time, the peptide is released when the polymer degrades. The graph in the lower left shows degradation as measured by absorbance of complexes with degradable polymers. R═CH₂CH₂ for hexanediol diacrylate (HDDA) and —OCH₂OCH₂O— for polyethyleneglycol diacrylate (PEGDA) having 0% HDDA (squares), 75% HDDA (circles), and 100% HDDA (triangles). See also Green et al., 2008.

### DETAILED DESCRIPTION

### I. General Considerations

The coronavirus SARS-CoV-2 infects host cells by binding to the angiotensin-converting enzyme 2 (ACE2) receptor which belongs to an anti-inflammatory, anti-thrombotic counter-regulatory arm of the renin-angiotensin system (RAS). ACE2 dysfunction and RAS dysregulation has been explored as a driving force in acute respiratory distress syndrome (ARDS) since the first severe acute respiratory syndrome coronavirus was detected almost 20 years ago. In vitro and in vivo studies utilizing animal models have been supportive of this hypothesis, but data from COVID-19 patients has been inconsistent and inconclusive.

We therefore sought to identify disruptions of the classical angiotensin-converting enzyme (ACE)/angiotensin [Ang] II/Ang II type-1 receptor (AT₁R) and the counter-regulatory ACE2/Ang 1-7/Mas Receptor (MasR) pathways in patients with COVID-19 and correlated these with severity of infection and markers of inflammation and coagulation. Ang II and Ang 1-7 were measured in plasma by ELISA in 230 patients, 166 who were COVID-19(+). Ang 1-7 was repressed in COVID-19 compared to SARS-CoV-2 negative controls. Furthermore, multivariable logistic regression analyses demonstrated that every 10 μg/mL increase of plasma Ang 1-7 was associated with a 3% reduction in odds of hospitalization (AOR 0.97, CI 0.95 to 0.99) and a 3% reduction in odds of requiring oxygen supplementation (AOR 0.97, CI 0.95 to 0.99) and/or ventilation (AOR 0.97, CI 0.94 to 0.99). Ang 1-7 was also inversely associated with pro-inflammatory cytokines and D-dimer in this patient cohort. These data suggest that reduced activity in this protective counter-regulatory arm of the RAS contribute to the hyper-immune response and diffuse coagulation activation documented in COVID-19.

Severe acute respiratory syndrome coronavirus 2 (SARS-CoV-2) causes a unique disease, COVID-19, that ranges in severity from asymptomatic to severe respiratory failure and death. Viral transmission throughout the world continues at a high rate despite the development and widespread use of effective vaccines. For those patients who contract COVID-19 and become severely ill, few therapeutic options have been shown to provide benefit and mortality rates are high. Additionally, the pathophysiology underlying COVID-19 disease presentation, progression and severity is incompletely understood. An aspect of the presently disclosed subject matter is in confirming the role of renin-angiotensin system dysfunction in the pathogenesis of COVID-19 in a large cohort of patients with diverse disease severity and outcomes. Additionally, to the knowledge of the present co-inventors, this is the first study to pair angiotensin peptide levels with inflammatory and thrombotic markers. These data support the role of ongoing clinical trials looking at renin-angiotensin system targeted therapeutics for the treatment of COVID-19.

### II. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

Following long-standing patent law convention, the terms “a”, “an”, and “the” refer to “one or more” when used in this application, including the claims. Thus, in some embodiments the phrase “a peptide” refers to one or more peptides.

The term “about”, as used herein to refer to a measurable value such as an amount of weight, time, dose (e.g., therapeutic dose), etc., is meant to encompass in some embodiments variations of ±20%, in some embodiments ±10%, in some embodiments ±5%, in some embodiments ±1%, in some embodiments ±0.5%, in some embodiments ±0.1%, and in some embodiments ±0.01% from the specified amount, as such variations are appropriate to perform the disclosed methods.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience. Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

A disease or disorder is “alleviated” if the severity of a symptom of the disease, condition, or disorder, or the frequency with which such a symptom is experienced by a subject, or both, are reduced.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” as used herein refers to an agent which enhances the pharmaceutical effect of another agent.

As used herein, the terms “administration of” and or “administering” a compound should be understood to mean providing a compound of the presently disclosed subject matter or a prodrug of a compound of the presently disclosed subject matter to a subject in need of treatment.

As used herein, the term “aerosol” refers to suspension in the air. In particular, aerosol refers to the particlization or atomization of a formulation of the presently disclosed subject matter and its suspension in the air.

As used herein, an “analog” of a chemical compound is a compound that, by way of example, resembles another in structure but is not necessarily an isomer (e.g., 5-fluorouracil is an analog of thymine).

As used herein, “amino acids” are represented by the full name thereof, by the three letter code corresponding thereto, or by the one-letter code corresponding thereto, as indicated in the following Table.

TABLE of
Amino Acids, Codes, and Functionally Equivalent
Codons

| | 3- | 1- | |
|---|---|---|---|
| | Letter | Letter | Functionally |
| Full Name | Code | Code | Equivalent Codons |
| Aspartic Acid | Asp | D | GAC GAU |
| Glutamic Acid | Glu | E | GAA GAG |
| Lysine | Lys | K | AAA AAG |
| Arginine | Arg | R | AGA AGG CGA CGC CGG CGU |
| Histidine | His | H | CAC CAU |
| Tyrosine | Tyr | Y | UAC UAU |
| Cysteine | Cys | C | UGC UGU |
| Asparagine | Asn | N | AAC AAU |
| Glutamine | Gln | Q | CAA CAG |
| Serine | Ser | S | ACG AGU UCA UCC UCG UCU |
| Threonine | Thr | T | ACA ACC ACG ACU |
| Glycine | Gly | G | GGA GGC GGG GGU |
| Alanine | Ala | A | GCA GCC GCG GCU |
| Valine | Val | V | GUA G?C GUG GUU |
| Leucine | Leu | L | UUA UUG CUA CUC CUG CUU |
| Isoleucine | Ile | I | AUA AUC AUU |
| Methionine | Met | M | AUG |
| Proline | Pro | P | CCA CCC CCG CCU |
| Phenylalanine | Phe | F | UUC UUU |
| Tryptophan | Trp | W | UGG |

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as exchanges within one of the following five groups:
I. Small aliphatic, nonpolar, or slightly polar residues: Ala, Ser, Thr, Pro, Gly;
II. Polar, negatively charged residues and their amides: Asp, Asn, Glu, Gin;
III. Polar, positively charged residues: His, Arg, Lys;
IV. Large, aliphatic, nonpolar residues: Met Leu, Ile, Val, Cys
V. Large, aromatic residues: Phe, Tyr, Trp

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antimicrobial agents” as used herein refers to any naturally-occurring, synthetic, or semi-synthetic compound or composition or mixture thereof, which is safe for human or animal use as practiced in the methods of this presently disclosed subject matter, and is effective in killing or substantially inhibiting the growth of microbes. “Antimicrobial” as used herein, includes antibacterial, antifungal, and antiviral agents.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens. DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the peptides encompasses natural or synthetic portions of a longer peptide or protein that are capable of specific binding to their natural ligand or of performing the desired function of the protein, for example, a fragment of a protein of larger peptide which still contains the epitope of interest and is immunogenic.

The term “biological sample”, as used herein, refers to samples obtained from a subject, including, but not limited to, skin, hair, tissue, blood, plasma, cells, sweat and urine.

As used herein, the term “carrier molecule” refers to any molecule that is chemically conjugated to an entity of interest, such as but not limited to a peptide of interest, including but not limited to an Ang (1-7) peptide or an analog or derivative thereof.

As used herein, the term “chemically conjugated”, or “conjugating chemically” refers to linking a chemical entity of interest, such as a peptide of interest, including but not limited to an Ang (1-7) peptide or an analog or derivative thereof, to the carrier molecule. This linking can occur on the genetic level using recombinant technology, wherein a hybrid protein may be produced containing the amino acid sequences, or portions thereof, of both the chemical entity of interest, such as a peptide of interest, including but not limited to an Ang (1-7) peptide or an analog or derivative thereof, and the carrier molecule. This hybrid protein is produced by an oligonucleotide sequence encoding both a chemical entity of interest, such as a peptide of interest, including but not limited to an Ang (1-7) peptide or an analog or derivative thereof, and the carrier molecule, or portions thereof. This linking also includes covalent bonds created between a chemical entity of interest, such as a peptide of interest, including but not limited to an Ang (1-7) peptide or an analog or derivative thereof, and the carrier protein using other chemical reactions, such as, but not limited to glutaraldehyde reactions. Covalent bonds may also be created using a third molecule bridging a chemical entity of interest, such as a peptide of interest, including but not limited to an Ang (1-7) peptide or an analog or derivative thereof, to the carrier molecule. These cross-linkers are able to react with groups, such as but not limited to, primary amines, sulfhydryls, carbonyls, carbohydrates, or carboxylic acids, on a chemical entity of interest, such as an antigen, and the carrier molecule. Chemical conjugation also includes non-covalent linkage between the a peptide of interest, including but not limited to an Ang (1-7) peptide or an analog or derivative thereof and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and in some embodiments at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, isolated nucleic acid, or other agent used in the method of the presently disclosed subject matter or to any type of substance or agent that is commonly considered a chemical, drug, or a candidate for use as a drug, as well as combinations and mixtures of the above. The term compound further encompasses molecules such as peptides and nucleic acids.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell is a cell being examined.

A “pathoindicative” cell is a cell which, when present in a tissue, is an indication that the animal in which the tissue is located (or from which the tissue was obtained) is afflicted with a disease or disorder.

A “pathogenic” cell is a cell which, when present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

As used herein, a “derivative” of a peptide refers to a peptide or other compound that may be produced from a peptide or other compound of similar structure in one or more steps. In some embodiments, a derivative of a peptide is a conjugate of the peptide, wherein the peptide is conjugated to another moiety.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting a risk or propensity to an addictive related disease disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular, and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription. The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly at least five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95%, and in some embodiments at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be in some embodiments at least about 20 nucleotides in length, in some embodiments at least about 50 nucleotides, in some embodiments from about 50 to about 100 nucleotides, in some embodiments at least about 100 to about 200 nucleotides, in some embodiments at least about 200 nucleotides to about 300 nucleotides, in some embodiments at least about 300 to about 350, in some embodiments at least about 350 nucleotides to about 500 nucleotides, in some embodiments at least about 500 to about 600, in some embodiments at least about 600 nucleotides to about 620 nucleotides, in some embodiments at least about 620 to about 650, and in some embodiments the nucleic acid fragment will be greater than about 650 nucleotides in length.

The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3TATGGC share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993. This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

By the term “immunizing a subject against an antigen” is meant administering to the subject a composition, a protein complex, a DNA encoding a protein complex, an antibody or a DNA encoding an antibody, which elicits an immune response in the subject, and, for example, provides protection to the subject against a disease caused by the antigen or which prevents the function of the antigen.

The term “immunologically active fragments thereof” will generally be understood in the art to refer to a fragment of a polypeptide antigen comprising at least an epitope, which means that the fragment at least comprises 4 contiguous amino acids from the sequence of the polypeptide antigen. As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit”, as used herein when referring to a function, refers to the ability of a compound of the presently disclosed subject matter to reduce or impede a described function. In some embodiments, inhibition is by at least 10%, in some embodiments by at least 25%, in some embodiments by at least 50%, and in some embodiments, the function is inhibited by at least 75%. When the term “inhibit” is used more generally, such as “inhibit Factor I”, it refers to inhibiting expression, levels, and activity of Factor I.

The term “inhibit a complex”, as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting, or applying, or administering” includes administration of a compound of the presently disclosed subject matter by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, or rectal approaches.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container which contains the identified compound presently disclosed subject matter or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a chemical entity, e.g., a molecule, that joins two other molecules either covalently or noncovalently, such as but not limited to, through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels

The term “nasal administration” in all its grammatical forms refers to administration of at least one compound of the presently disclosed subject matter through the nasal mucous membrane to the bloodstream for systemic delivery of at least one compound of the presently disclosed subject matter. The advantages of nasal administration for delivery are that it does not require injection using a syringe and needle, it avoids necrosis that can accompany intramuscular administration of drugs, trans-mucosal administration of a drug is highly amenable to self administration, and intranasal administration of antigens exposes the antigen to a mucosal compartment rich in surrounding lymphoid tissues, which can promote the development of a more potent immune response, particularly more potent mucosal immune responses.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”. “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand” sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U. G. C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides but when used in the context of a longer amino acid sequence can also refer to a longer polypeptide.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application. As such, As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the U.S. Federal government or listed in the U.S. Pharmacopeia for use in an animal. In some embodiments, a pharmaceutically acceptable carrier is pharmaceutically acceptable for use in a human.

As used herein, “pharmaceutical compositions” include formulations for human and veterinary use.

“Plurality” means at least two.

The terms “polymer” and “polymeric” refer to chemical structures that have repeating units (i.e., multiple copies of a given chemical substructure). As used herein, polymers can refer to groups having more than 5 or more than 10 repeating units and/or to groups wherein the repeating unit is other than methylene. Polymers can be formed from polymerizable monomers. A polymerizable monomer is a molecule that comprises one or more reactive moieties e.g., siloxy ethers, hydroxyls, amines, vinylic groups (i.e., carbon-carbon double bonds), halides (i.e., Cl, Br, F, and 1), esters, activated esters, and the like that can react to form bonds with other molecules. Generally, each polymerizable monomer molecule can bond to two or more other molecules. In some cases, a polymerizable monomer will bond to only one other molecule, forming a terminus of the polymeric material. Some polymers contain biodegradable linkages, such as esters or amides, such that they can degrade overtime under biological conditions.

A “copolymer” refers to a polymer derived from more than one species of monomer. A “random copolymer” refers to a copolymers where the sequential distribution of the monomeric units derived from different monomers is random.

As used herein, a “block copolymer” refers to a copolymer that comprises blocks (i.e., polymeric sub-sections of the whole copolymer) in a linear sequence. A “block” refers to a portion of a copolymer that has at least one feature that is not present in the adjacent portions of the macromolecule. Thus, a “block copolymer” can refer to a copolymer in which adjacent blocks are constitutionally different, i.e., each of these blocks comprises constitutional units derived from different characteristic species of monomer or with different composition or sequence distribution of constitutional units.

For example, a diblock copolymer of PEG and polystyrene can be referred to as PEG-block-polystyrene. Such a copolymer can also be referred to generically as an “AB block copolymer.” Likewise, a triblock copolymer can be represented as “ABA.” Other types of block polymers exist, such as multiblock copolymers of the (AB)n type, ABC block polymers comprising three different blocks, and star block polymers, which have a central point with three or more arms, each of which is in the form of a block copolymer, usually of the AB type.

As used herein, a “graft macromolecule” or “graft polymer” refers to a macromolecule comprising one or more species of block connected to the main chain as side chains, wherein the side chains comprise constitutional or configurational features that differ from those in the main chain.

A “branch point” (or “junction point”) refers to a point on a chain (e.g., a main chain) at which a branch is attached. A “branch,” also referred to as a “side chain,” “graft,” or “pendant chain,” is an oligomeric or polymeric offshoot from a macromolecule chain. An oligomeric branch can be termed a “short chain branch,” whereas a polymeric branch can be termed a “long chain branch.”

A “chain” refers to the whole or part of a polymer, an oligomer, or a block comprising a linear or branched sequence of constitutional units between two boundary constitutional units, wherein the two boundary constitutional units can comprise an end group, a branch point, or combinations thereof.

A “main chain” or “backbone” refers to a linear chain from which all other chains are regarded as being pendant.

A “side chain” refers to a linear chain which is attached to a main chain at a branch point.

An “end group” (or “terminal group”) refers to a constitutional unit that comprises the extremity of a macromolecule or oligomer and, by definition, is attached to only one constitutional unit of a macromolecule or oligomer.

“Biocompatible” as used herein, generally refers to a material and any metabolites or degradation products thereof that are generally non-toxic to the recipient and do not cause any significant adverse effects to the recipient.

Biocompatible, relatively non-biodegradable polymers include, but are not limited to, polyacrylates, polymethacryates, polyureas, polyurethanes, polyolefins, polyvinylhalides, polyvinylidenehalides, polyvinylethers, polyvinylaromatics, polyvinylesters, polyacrylonitriles, alkyd resins, polysiloxanes and epoxy resins.

“Biodegradable” and “biologically degradable” as used herein, generally refer to a material that will degrade or erode under physiologic conditions to smaller units or chemical species that are capable of being metabolized, eliminated, or excreted by the subject. In some embodiments, the degradation time is a function of polymer composition and morphology. Suitable degradation times are from days to weeks. For example, in some embodiments, the polymer can degrade over a time period from seven days to 24 weeks, optionally seven days to twelve weeks, optionally from seven days to six weeks, or further optionally from seven days to three weeks. Biodegradable polymers can be synthetic, semi-synthetic or natural polymers. Exemplary naturally-occurring biocompatible, biodegradable polymers include, but are not limited to, collagen, chitosan, alginate, fibrin, fibrinogen, cellulosics, starches, dextran, dextrin, hyaluronic acid, heparin, glycosaminoglycans, polysaccharides and elastin.

The term “hydrophilic” can refer to a group that dissolves or preferentially dissolves in water and/or aqueous solutions. The term “hydrophilic polymer” as used herein generally refers to hydrophilic organic polymers, such as but not limited to, polyvinylpyrrolidone (PVP); poly(vinyl alcohol); polyvinvlmethylether, polvmethyloxazoline; polyethyloxazolinc polyhydroxy-propyloxazoline; poly(acrylic acids) and poly(acrvlamides), such as, but not limited to, polyhydroxypropylmethacrylamide, polymethyacrylamide, polydimethylacrylamide, polyhydroxylpropylmethacrylate, and polyhydroxy-ethylacrylate; hydroxymethylcellulose; hydroxyethylcellulose; polyethylene-imine (PEI); polyethylene glycol (i.e., PEG) or another hydrophilic poly(alkyleneoxide); polyglycerine; and polyaspartamide. The term “hydrophilic” refers to the ability of a molecule or chemical species to interact with water.

The term “hydrophobic” refers to groups that do not significantly dissolve in water and/or aqueous solutions and/or which preferentially dissolves in fats and/or non-aqueous solutions. Non-limiting examples of hydrophobic polymers include polycaprolactam, poly(lactic acid), poly(glycolic acid), polycaprolactone, PLGA or co-polymers, or combinations of any two or more of them.

The term “amphiphilic” refers to a molecule or polymer that contains both hydrophilic and hydrophobic groups.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of contracting the disease and/or developing a pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds to”, as used herein, is meant when a compound or ligand functions in a binding reaction or assay conditions which is determinative of the presence of the compound in a sample of heterogeneous compounds.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, the term “subject” refers to an individual (e.g., human, animal, or other organism) to be assessed, evaluated, and/or treated by the methods or compositions of the presently disclosed subject matter. Subjects include, but are not limited to, mammals (e.g., murines, simians, equines, bovines, porcines, canines, felines, and the like), and includes humans. As used herein, the terms “subject” and “patient” are used interchangeably, unless otherwise noted.

As used herein, a “subject in need thereof” is a patient, animal, mammal, or human, who will benefit from the methods and compositions of the presently disclosed subject matter.

As used herein, “substantially homologous amino acid sequences” includes those amino acid sequences which have in some embodiments at least about 95% homology, in some embodiments at least about 96% homology, in some embodiments at least about 97% homology, in some embodiments at least about 98% homology, and in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is in some embodiments at least about 50%, 65% 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: in some embodiments 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in IX SSC, 0.1% SDS at 50° C.; in some embodiments 7% SDS, 0.5 M NaPO₄, 1 mM EDTA at 50° C. with washing in 0.5×SSC, 0.1% SDS at 50° C.; and in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when it is in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

The term “modulate”, as used herein, refers to changing the level of an activity, function, or process. The term “modulate” encompasses both inhibiting and stimulating an activity, function, or process.

### III. Methods and Uses

The presently disclosed subject matter is based on the presently disclosed discoveries that high levels of the peptide Ang II were associated with mortality from COVID-19; low levels of the peptide Ang 1-7 were associated with COVID-19 infection and severe outcome indicators including hospitalization, oxygen supplementation, and ventilation; increased ratios of Ang II:Ang (1-7) were associated with severe outcome indicators including hospitalization, oxygen supplementation, ventilation and mortality; Ang II levels were positively correlated with D-dimer and pro-inflammatory cytokines, including IL-1β; and Ang (1-7) levels were negatively correlated with D-dimer and pro-inflammatory cytokines, including IL-6, TNFα, and M-CSF.

Thus, in some embodiments the presently disclosed subject matter relates to methods for treating subjects with coronavirus infections and consequences therefrom. In some embodiments, the presently disclosed methods comprise, consist essentially of, or consist of providing a subject infected with a coronavirus resulting in and/or having a prothrombotic condition in addition to being infected by a coronavirus; and administering to the subject an angiotensin (1-7) peptide or an analog or derivative thereof, a Mas Receptor (MasR) agonist, or any combination thereof. In some embodiments, the coronavirus is a SARS-CoV-1, MERS, or SARS-CoV-2 coronavirus. As such, in some embodiments the subject is suffering from COVID-19 disease.

Alternatively or in addition, a composition comprising, consisting essentially of, or consisting of an ACE2 polypeptide, optionally a recombinant ACE2 polypeptide, and/or a nucleic acid sequence encoding the same could be administered to a subject in order to increase endogenous Ang (1-7) to modulate an imbalance of Ang II to Ang (1-7) in the subject that results from a coronavirus infection or any other etiology. The administration can be accomplished by administering a vector encoding the polypeptide and comprising regulatory sequences, including a promoter, such as a promoter as defined herein, operably linked to a coding sequence or any other gene therapy or other therapeutic approach as would be apparent to one of ordinary skill in the art upon a review of the instant disclosure. In some embodiments, the ACE2 polypeptide is a human ACE2 polypeptide (Expasy ENZYME Entry EC 3.4.17.23) or a biologically active fragment thereof. In some embodiments, the human ACE2 polypeptide has an amino acid sequence as set forth in any of Accession Nos. NP_068576.1 (SEQ ID NO: 6), NP_001358344.1 (SEQ ID NO: 8), NP_001373188.1 (SEQ ID NO: 10), NP_001373189.1 (SEQ ID NO: 12), NP_001375381.1 (SEQ ID NO: 14), and NP_001376331.1 (SEQ ID NO: 16) of the GENBANK® biosequence database or a functional derivative (e.g., alternative transcription and/or translation variant) thereof. In some embodiments, the human ACE2 polypeptide is encoded by a nucleotide sequence as set forth in any of Accession Nos. NM_021804.3 (SEQ ID NO: 5), NM_001371415.1 (SEQ ID NO: 7), NM_001386259.1 (SEQ ID NO: 9), NM_001386260.1 (SEQ ID NO: 11), NM_001388452.1 (SEQ ID NO: 13), and NM_001389402.1 (SEQ ID NO: 15) of the GENBANK® biosequence database or a functional derivative (e.g., alternative transcription and/or translation variant) thereof. See also U.S. Patent Application Publication No. 2018/0289779 and PCT International Patent Application Publication No. WO 2021/174107.

As used herein, the phrase “an angiotensin (1-7) peptide” (abbreviated “Ang (1-7)”, “Ang(1-7)”, “Ang 1-7”, etc.) refers to any peptide, peptidomimetic, or analog or derivative thereof that has one or more of the biological activities of a naturally occurring angiotensin (1-7) peptide. In some embodiments, the one or more of the biological activities of the naturally occurring Ang (1-7) peptide relate to its binding to the Mas receptor (MasR), which results in anti-inflammation, vasodilation, anti-fibrosis, and anti-thrombosis. In this context, an Ang (1-7) peptide, analog, or derivative thereof counteracts the activities of angiotensin (1-8) peptides via binding to the Angiotensin II type-1 receptor (AT₁R), which is associated with inflammation, vasoconstriction, aldosterone production, and thrombosis. Ang (1-7) peptides include those comprising, consisting essentially of, of consisting of the amino acid sequences Asp-Arg-Val-Tyr-Ile-His-Pro (SEQ ID NO: 1), Asp-Arg-Val-Ser-Ile-His-Cys (SEQ ID NO: 2), and Ala-Arg-Val-Ser-Ile-His-Cys (SEQ ID NO: 3), as well as functional equivalents thereof.

In some embodiments, the Mas Receptor (MasR) agonist is N-(Ethylcarbamoyl)-3-(4-((5-formyl-4-methoxy-2-phenyl-1H-imidazol-1-YL)methyl)phenyl)-5-isobutylthiophene-2-sulfonamide (AVE0991), an analog thereof, and/or a derivative thereof. Other MasR agonists would be apparent to one of ordinary skill in the art upon a review of the instant disclosure.

Given Ang (1-7)'s role in inhibiting thrombosis, in some embodiments the presently disclosed subject matter relates to preventing and/or treating undesirable thrombosis, including but not limited to thrombosis incident to coronavirus infection. Thus, in some embodiments a subject for whom the presently disclosed methods and uses are appropriate is a subject that has and/or is at risk for developing a thrombotic complication, optionally a thrombotic complication selected from the group consisting of acute limb ischemia, abdominal and/or thoracic aortic thrombosis, mesenteric ischemia, myocardial infarction, venous thromboembolism, pulmonary embolism, cerebrovascular accident, and any form of systemic arterial embolism, either as a consequence of or concurrently with a corona virus infection.

Similarly, in some embodiments a subject for whom the instant methods and uses are appropriate is a subject who has an elevated risk of an adverse pregnancy outcome. The phrase “adverse pregnancy outcome” refers in some embodiments to intrauterine fetal death, stillbirth, fetal growth restriction, and preterm birth secondary to placental insufficiency. In some embodiments, the adverse pregnancy outcome results from and/or is secondary to thrombosis formation in the subject associated with and/or exacerbated by a coronavirus infection.

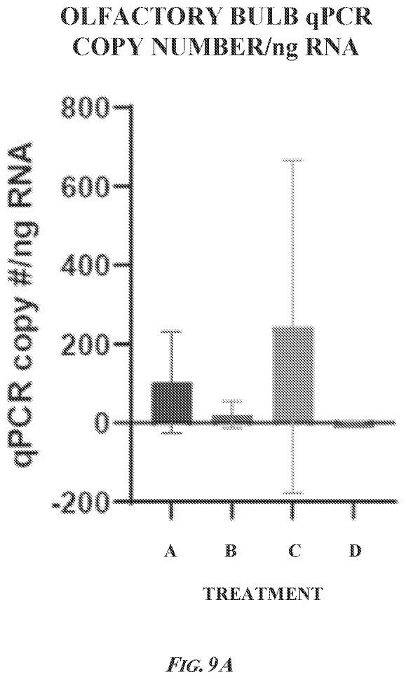

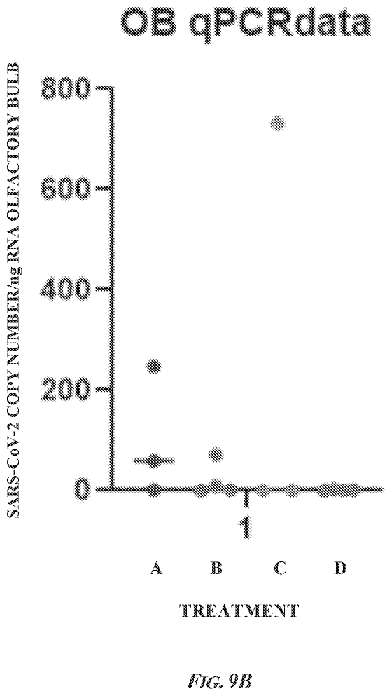

In some embodiments of the presently disclosed methods, the subject is suffering from weight loss and/or olfactory nerve invasion by the coronavirus. See FIGS. 8, 9A, and 9B.

Thus, in some embodiments the subject is at risk of a complication resulting from the coronavirus infection due to an underlying prothrombotic state, which optionally results from pregnancy, malignancy, a genetic condition, and/or a rheumatologic condition that predisposes the subject to thrombosis formation.

In some embodiments, a method of the presently disclosed subject matter relates to administering an Ang (1-7) peptide or an analog or derivative thereof, a Mas Receptor (MasR) agonist, or any combination thereof parenterally, rectally, orally, or any combination thereof. Exemplary parenteral administration routes include, but are not limited to intravenous, subcutaneous, inhalation, intradermal, transdermal, and/or transmucosal administration.

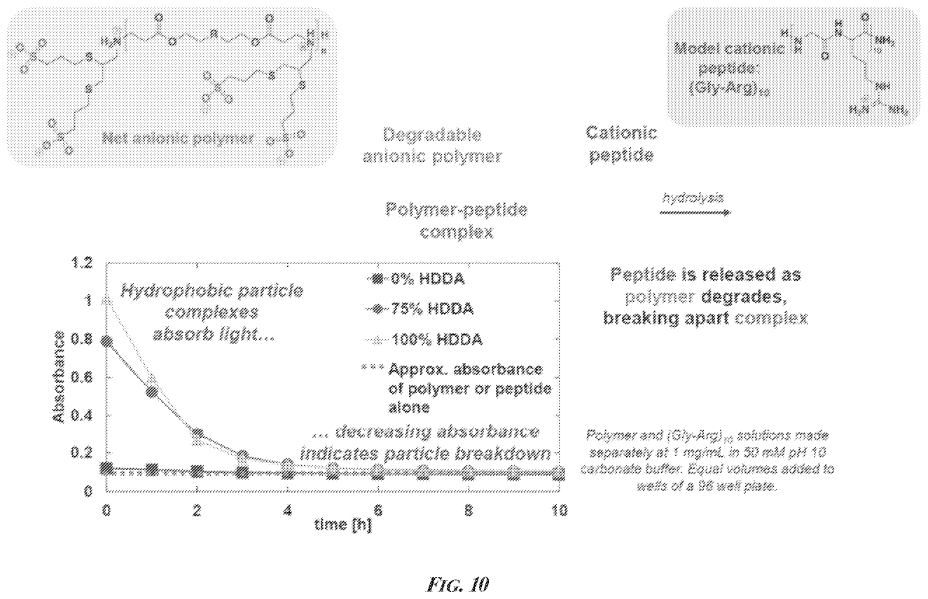

In some embodiments, a composition of the presently disclosed subject matter comprises, consists essentially of, or consists of an Ang (1-7) peptide or an analog or derivative thereof, a Mas Receptor (MasR) agonist, or any combination thereof completed to or otherwise associated with a polymer. In some embodiments, the polymer is non-degradable. In some embodiments, the polymer is a degradable (e.g., biologically degradable) polymer. In some embodiments, the polymer is an anionic polymer. An exemplary polymer is shown in FIG. 10. For example, the polymer can be a poly(β-amino ester) prepared by polymerization of one or more diacrylates (e.g., 1,6-hexanediol diacrylate (HDDA) and/or poly(ethylene glycol) diacrylate (PEGDA)) with one or more amines, where the polymer can further include pendant or grafted functional groups that are anionic at relevant biological pH, such as, but not limited to carboxylic acid groups or sulfate groups, such as shown in the polymer of FIG. 10. The hydrophobicity of the polymer can be controlled by varying the amount of diacrylate. In some embodiments, the anionic groups can be incorporated, for example, using mercaptopropionic acid or a thiolated anionic peptide.

In some embodiments, the Ang (1-7) peptide or an analog or derivative thereof, the Mas Receptor (MasR) agonist, or the combination thereof and the polymer are associated with each other via an electrostatic interaction, a hydrophobic interaction, a hydrogen bonding interaction, or any combination thereof. In some embodiments, the polymer is a block copolymer comprising a neutral, hydrophilic block (e.g., PEG) and a block that can interact with the Ang(1-7) peptide or an analog or derivative thereof. For example, the interacting block of the block copolymer can include pyridine pendant groups to interact with the tyrosine phenol group in the Ang(1-7) peptide or analog or derivative thereof via hydrogen bonding. An exemplary material for such an interacting block is a random copolymer of styrene and 2-vinyl pyridine. An exemplary polymeric interacting block that can interact via hydrophobic interactions (e.g., with valine, isoleucine, and tyrosine residues on the Ang(1-7) peptide) is polystyrene. Additional hydrophobic polymers include, but are not limited to polyolefins, such as polyethylene; polyurethanes; polysiloxanes (e.g., polydimethylsiloxane), acrylics, and epoxies. Blocks for electrostatic interactions (i.e., with the arginine and histidine residues of the Ang(1-7) peptide) include polymer chains with pendant carboxylate and/or sulfonate groups, such as those described for FIG. 10.

In some embodiments, the Ang (1-7) peptide or an analog or derivative thereof, the Mas Receptor (MasR) agonist, or the combination thereof and the polymer are associated with each other via an electrostatic interaction, a hydrophobic interaction, a hydrogen bonding interaction, or any combination thereof.

In some embodiments, the presently disclosed subject matter relates to a combination therapy, in which a subject in need thereof is administered an Ang (1-7) peptide or an analog or derivative thereof and/or a Mas Receptor (MasR) agonist, and/or a combination thereof and also one or more additional therapies including but not limited to a therapy designed to inhibit a biological activity of an angiotensin-converting enzyme (ACE). Exemplary ACE inhibitors include benazepril, captopril, enalapril, fosinopril, lisinopril, moexioril, perindopril, quinapril, ramipril, and trandolapril.

In some embodiments, the presently disclosed subject matter relates to a combination therapy, in which a subject in need thereof is administered an Ang (1-7) peptide or an analog or derivative thereof and/or a Mas Receptor (MasR) agonist, and/or a combination thereof and also one or more additional therapies including but not limited to a therapy designed to inhibit a biological activity of an Angiotensin II Receptor. Inhibitors of Angiotensin II Receptors are referred to as Angiotensin II Receptor Blockers (ARBs). In some embodiments, an ARB is selected from the group consisting of candesartan, irbesartan, olmesartan, losartan, valsartan, telmisartan, and eprosartan. As would be recognized by one of ordinary skill in the art, in some embodiments the combination therapy comprises, consists essentially of, or consists of treating a subject in need thereof with an Ang (1-7) peptide or an analog or derivative thereof and/or a Mas Receptor (MasR) agonist, and also at least one of an ACE inhibitor and/or an ARB.

In addition to the methods described herein, in some embodiments the presently disclosed subject matter relates to uses of angiotensin (1-7) peptides and/or analogs or derivatives thereof and/or Mas Receptor (MasR) agonists for the preparation of medicaments for treating subjects infected with a coronavirus. In some embodiments, the subject is a subject infected with a coronavirus and having a prothrombic condition in addition to being infected by a coronavirus.

In some embodiments, the presently disclosed subject matter relates to uses of angiotensin (1-7) peptides and/or analogs or derivatives thereof and/or Mas Receptor (MasR) agonists for treating subjects infected with a coronavirus. In some embodiments, the subject is a subject infected with a coronavirus and having a prothrombic condition in addition to being infected by a coronavirus.

In some embodiments of the presently disclosed uses, the angiotensin (1-7) peptide and/or the analog or derivative thereof is provided in a composition comprising a degradable polymer having an electrostatic interaction with the angiotensin (1-7) peptide and/or the analog or derivative thereof, a non-degradable polymer having an electrostatic interaction with the angiotensin (1-7) peptide and/or the analog or derivative thereof, a non-degradable polymer having a hydrophobic interaction with the angiotensin (1-7) peptide and/or the analog or derivative thereof, a non-degradable polymer having a hydrogen bonding interaction with the angiotensin (1-7) peptide and/or the analog or derivative thereof, or any combination thereof.

In some embodiments of the presently disclosed uses, the subject is suffering from weight loss and/or olfactory nerve invasion by the coronavirus. See FIGS. 8, 9A, and 9B.

### IV. Compositions

In some embodiments, the presently disclosed subject matter relates to compositions for use in the methods and uses disclosed herein. As such, in some embodiments the presently disclosed subject matter relates to compositions comprising, consisting essentially of, or consisting of one or more Ang (1-7) peptides, analogs thereof, derivatives thereof, and/or MasR agonists.

In some embodiments, a composition of the presently disclosed subject matter comprises, consists essentially of, or consists of one or more Ang (1-7) peptides, analogs thereof, derivatives thereof, and/or MasR agonists complexed to and/or otherwise associated with one or more polymers.

In some embodiments, the one or more polymers include at least one degradable (e.g., biodegradable) polymer. In some embodiments, the one or more polymers do not include any degradable (e.g., biodegradable) polymers.

In some embodiments, the one or more Ang (1-7) peptides, analogs thereof, derivatives thereof, and/or MasR agonists and the one or more polymers interact via an electrostatic interaction, a hydrophobic interaction, a hydrogen bonding interaction, or any combination thereof.

In some embodiments, the composition is formulated for the treatment of a subject infected with a coronavirus and having a prothrombic condition in addition to being infected by a coronavirus.

In some embodiments, the subject is suffering from weight loss and/or olfactory nerve invasion by the coronavirus. See FIGS. 8, 9A, and 9B.

### IV.A. Pharmaceutical Compositions

In some embodiments, the compositions of the presently disclosed subject matter are provided as part of a pharmaceutical composition. As used herein, the term “pharmaceutical composition” refers to a composition comprising at least one active ingredient (e.g., an Ang (1-7) peptide or an analog or derivative thereof and/or a MasR agonist of the presently disclosed subject matter), whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

In some embodiments, a pharmaceutical composition of the presently disclosed subject matter comprises, consists essentially of, or consists of at least one active ingredient (e.g., an inhibitor of the presently disclosed subject matter) and a pharmaceutically acceptable diluent and/or excipient. As used herein, the term “pharmaceutically acceptable” refers to physiologically tolerable, for either human or veterinary application. Similarly, “pharmaceutical compositions” include formulations for human and veterinary use. The term “pharmaceutically acceptable carrier” also refers to a chemical composition with which an appropriate compound or derivative can be combined and which, following the combination, can be used to administer the appropriate compound to a subject. In some embodiments, a pharmaceutically acceptable diluent and/or excipient is pharmaceutically acceptable for use in a human.

In some embodiments, the pharmaceutical compositions of the presently disclosed subject matter are for use in preventing and/or treating a disease or disorder associated with genotoxic stress-induced cardiac toxicity in a subject in need thereof.

The pharmaceutical compositions of the presently disclosed subject matter can in some embodiments consist of the active ingredient alone, in a form suitable for administration to a subject, or the pharmaceutical composition can in some embodiments comprise or consist essentially of the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt refers to an ester or salt form of the active ingredient which is compatible with any other ingredients of the pharmaceutical composition, which is not deleterious to the subject to which the composition is to be administered.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts.

### IV.B. Formulations

The compositions of the presently disclosed subject matter thus comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

Controlled- or sustained-release formulations of a pharmaceutical composition of the presently disclosed subject matter can be made using conventional technology. A formulation of a pharmaceutical composition of the presently disclosed subject matter suitable for oral administration can be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin.

Liquid suspensions may further comprise one or more additional ingredients including, but not limited to, suspending agents, dispersing and/or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl parahydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the presently disclosed subject matter may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the presently disclosed subject matter may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the presently disclosed subject matter may also be prepared, packaged, or sold in the form of oil in water emulsion or a water-in-oil emulsion.

The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the presently disclosed subject matter may also be prepared, packaged, or sold in a formulation suitable for parenteral administration, including but not limited to intraocular injection.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane dial, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems.

Compositions for sustained release or implantation may comprise pharmaceutically acceptable polymeric or hydrophobic materials such as an emulsion, an ion exchange resin, a sparingly soluble polymer, or a sparingly soluble salt. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the presently disclosed subject matter may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, can in some embodiments have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oil vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Genaro, 1985, which is incorporated herein by reference in its entirety.

### IV.C. Administration

With regard to administering a composition of the presently disclosed subject matter, methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intravitreous administration, including via intravitreous sustained drug delivery device, intracameral (into anterior chamber) administration, suprachoroidal injection, subretinal administration, subconjunctival injection, sub-tenon administration, peribulbar administration, transscleral drug delivery, intraocular injection, intravenous injection, intraparenchymal/intracranial injection, intra-articular injection, retrograde ureteral infusion, intrauterine injection, intratesticular tubule injection, intrathecal injection, intraventricular (e.g., inside cerebral ventricles) administration, administration via topical eye drops, and the like. Administration can be continuous or intermittent. In some embodiments, a preparation can be administered therapeutically; that is, administered to treat an existing disease or condition. In some embodiments, a preparation can be administered prophylactically; that is, administered for prevention of a disease, disorder, or condition.

### IV.D. Doses

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

In some embodiments, a composition is administered in a therapeutically effective amount and/or according to a dosing regimen that is correlated with a particular desired outcome (e.g., with treating or reducing severity of a coronavirus infection or any consequence thereof). Particular doses or amounts to be administered in accordance with the presently disclosed subject matter can vary, for example, depending on the nature and/or extent of the desired outcome, on particulars of route and/or timing of administration, and/or on one or more characteristics (e.g., weight, age, personal history, genetic characteristic, lifestyle parameter, severity of pulmonary defect and/or level of risk of pulmonary defect, etc., or combinations thereof). Such doses or amounts can be determined by those of ordinary skill. In some embodiments, an appropriate dose or amount is determined in accordance with standard clinical techniques. For example, in some embodiments, an appropriate dose or amount is a dose or amount sufficient to reduce a disease severity index score by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more.

For example, in some embodiments, an appropriate dose or amount is a dose or amount sufficient to reduce a disease severity index score by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100%. Alternatively or additionally, in some embodiments, an appropriate dose or amount is determined through use of one or more in vitro or in vivo assays to help identify desirable or optimal dosage ranges or amounts to be administered.

In some embodiments, an Ang (1-7) peptide or MasR agonist is administered at a therapeutically effective amount. As used herein, the term “therapeutically effective amount” is largely determined based on the total amount of the therapeutic agent contained in the pharmaceutical compositions of the presently disclosed subject matter. Generally, a therapeutically effective amount is sufficient to achieve a meaningful benefit to the subject (e.g., treating, modulating, curing, preventing, and/or ameliorating the underlying disease, disorder, or condition). In some embodiments, appropriate doses or amounts to be administered may be extrapolated from dose-response curves derived from in vitro or animal model test systems. In some embodiments, a therapeutically effective amount is sufficient to achieve a meaningful anti-thrombotic benefit to a prothrombotic subject.

Therapeutically effective dosage amounts of Ang (1-7) peptides or MasR agonists, including derivatives, analogs, and/or salts, may be present in varying amounts in various embodiments. For example, in some embodiments, a therapeutically effective amount of an Ang (1-7) peptide can be an amount ranging from about 10-1000 mg (e.g., about 20 mg-1,000 mg, 30 mg-1,000 mg, 40 mg-1,000 mg, 50 mg-1,000 mg, 60 mg-1,000 mg, 70 mg-1,000 mg, 80 mg-1,000 mg, 90 mg-1,000 mg, about 10-900 mg, 10-800 mg, 10-700 mg, 10-600 mg, 10-500 mg, 100-1000 mg, 100-900 mg, 100-800 mg, 100-700 mg, 100-600 mg, 100-500 mg, 100-400 mg, 100-300 mg, 200-1000 mg, 200-900 mg, 200-800 mg, 200-700 mg, 200-600 mg, 200-500 mg, 200-400 mg, 300-1000 mg, 300-900 mg, 300-800 mg, 300-700 mg, 300-600 mg, 300-500 mg, 400 mg-1,000 mg, 500 mg-1,000 mg, 100 mg-900 mg, 200 mg-800 mg, 300 mg-700 mg, 400 mg-700 mg, and 500 mg-600 mg). In some embodiments, an Ang (1-7) peptide or MasR agonist is present in an amount of or greater than about 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg. In some embodiments, an Ang (1-7) peptide or MasR agonist is present in an amount of or less than about 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, or 100 mg. In some embodiments, the therapeutically effective amount described herein is provided in one dose. In some embodiments, the therapeutically effective amount described herein is provided in one day. [0140] In other embodiments, a therapeutically effective dosage amount may be, for example, about 0.001 mg/kg weight to 500 mg/kg weight, e.g., from about 0.001 mg/kg weight to 400 mg/kg weight, from about 0.001 mg/kg weight to 300 mg/kg weight, from about 0.001 mg/kg weight to 200 mg/kg weight, from about 0.001 mg/kg weight to 100 mg/kg weight, from about 0.001 mg/kg weight to 90 mg/kg weight, from about 0.001 mg/kg weight to 80 mg/kg weight, from about 0.001 mg/kg weight to 70 mg/kg weight, from about 0.001 mg/kg weight to 60 mg/kg weight, from about 0.001 mg/kg weight to 50 mg/kg weight, from about 0.001 mg/kg weight to 40 mg/kg weight, from about 0.001 mg/kg weight to 30 mg/kg weight, from about 0.001 mg/kg weight to 25 mg/kg weight, from about 0.001 mg/kg weight to 20 mg/kg weight, from about 0.001 mg/kg weight to 15 mg/kg weight, from about 0.001 mg/kg weight to 10 mg/kg weight. In some embodiments, the therapeutically effective amount described herein is provided in one dose. In some embodiments, the therapeutically effective amount described herein is provided in one day.

In some embodiments, a therapeutically effective dosage amount can be, for example, about 0.001 mg/kg weight to about 1 mg/kg weight, e.g. from about 0.001 mg/kg weight to about 0.9 mg/kg weight, from about 0.001 mg/kg weight to about 0.8 mg/kg weight, from about 0.001 mg/cg weight to about 0.8 mg/kg weight, from about 0.001 mg/kg weight to about 0.7 mg/kg weight, from about 0.001 mg/kg weight to about 0.6 mg/kg weight, from about 0.001 mg/kg weight to about 0.5 mg/kg weight, from about 0.01 mg/kg weight to about 1 mg/kg weight, from about 0.01 mg/kg weight to about 0.9 mg/kg weight, from about 0.01 mg/kg weight to about 0.8 mg/kg weight, from about 0.01 mg/kg weight to about 0.7 mg/kg weight, from about 1.1 mg/kg weight to about 0.6 mg/kg weight, from about 0.01 mg/kg weight to about 0.5 mg/kg weight, from about 0.02 mg/kg weight to about 1 mg/kg weight, from about 0.02 mg/kg weight to about 0.9 mg/kg weight, from about 0.02 mg/kg weight to about 0.8 mg/kg weight, from about 0.02 mg/kg weight to about 0.7 mg/kg weight, from about 0.02 mg/kg weight to about 0.6 mg/kg weight, from about 0.02 mg/kg weight to about 0.5 mg/kg weight, from about 0.03 mg/kg weight to about 1 mg/kg weight, from about 0.03 mg/kg weight to about 0.9 mg/kg weight, from about 0.03 mg/kg weight to about 0.8 mg/kg weight, from about 0.03 mg/kg weight to about 0.7 mg/kg weight, from about 0.03 mg/kg weight to about 0.6 mg/kg weight, from about 0.03 mg/kg weight to about 0.5 mg/kg weight, from about 0.04 mg/kg weight to about 1 mg/kg weight, from about 0.04 mg/kg weight to about 0.9 mg/kg weight, from about 0.04 mg/kg weight to about 0.8 mg/kg weight, from about 0.04 mg/kg weight to about 0.7 mg/kg weight, from about 0.04 mg/kg weight to about 0.6 mg/kg weight, from about 0.04 mg/kg weight to about 0.5 mg/kg weight, from about 0.05 mg/kg weight to about 1 mg/kg weight, from about 0.05 mg/kg weight to about 0.9 mg/kg weight, from about 0.05 mg/kg weight to about 0.8 mg/kg weight, from about 0.05 mg/kg weight to about 0.7 mg/kg weight, from about 0.05 mg/kg weight to about 0.6 mg/kg weight, from about 0.05 mg/kg weight to about 0.5 mg/kg weight. In some embodiments, the therapeutically effective amount described herein is provided in one dose. In some embodiments, the therapeutically effective amount described herein is provided in one day.

In some embodiments, a therapeutically effective dosage amount may be, for example, about 0.0001 mg/kg weight to 0.1 mg/kg weight, e.g. from about 0.0001 mg/kg weight to 0.09 mg/kg weight, from about 0.0001 mg/kg weight to 0.08 mg/kg weight, from about 0.0001 mg/kg weight to 0.07 mg/kg weight, from about 0.0001 mg/kg weight to 0.06 mg/kg weight, from about 0.0001 mg/kg weight to 0.05 mg/kg weight, from about 0.0001 mg/kg weight to about 0.04 mg/kg weight, from about 0.0001 mg/kg weight to 0.03 mg/kg weight, from about 0.0001 mg/kg weight to 0.02 mg/kg weight, from about 0.0001 mg/kg weight to 0.019 mg/kg weight, from about 0.0001 mg/kg weight to 0.018 mg/kg weight, from about 0.0001 mg/kg weight to 0.017 mg/kg weight, from about 0.0001 mg/kg weight to 0.016 mg/kg weight, from about 0.0001 mg/kg weight to 0.015 mg/kg weight, from about 0.0001 mg/kg weight to 0.014 mg/kg weight, from about 0.0001 mg/kg weight to 0.013 mg/kg weight, from about 0.0001 mg/kg weight to 0.012 mg/kg weight, from about 0.0001 mg/kg weight to 0.011 mg/kg weight, from about 0.0001 mg/kg weight to 0.01 mg/kg weight, from about 0.0001 mg/kg weight to 0.009 mg/kg weight, from about 0.0001 mg/kg weight to 0.008 mg/kg weight, from about 0.0001 mg/kg weight to 0.007 mg/kg weight, from about 0.0001 mg/kg weight to 0.006 mg/kg weight, from about 0.0001 mg/kg weight to 0.005 mg/kg weight, from about 0.0001 mg/kg weight to 0.004 mg/kg weight, from about 0.0001 mg/kg weight to 0.003 mg/kg weight, from about 0.0001 mg/kg weight to 0.002 mg/kg weight. In some embodiments, the therapeutically effective dose may be 0.0001 mg/kg weight, 0.0002 mg/kg weight, 0.0003 mg/kg weight, 0.0004 mg/kg weight, 0.0005 mg/kg weight, 0.0006 mg/kg weight, 0.0007 mg/kg weight, 0.0008 mg/kg weight, 0.0009 mg/kg weight, 0.001 mg/kg weight, 0.002 mg/kg weight, 0.003 mg/kg weight, 0.004 mg/kg weight, 0.005 mg/kg weight, 0.006 mg/kg weight, 0.007 mg/kg weight, 0.008 mg/kg weight, 0.009 mg/kg weight, 0.01 mg/kg weight, 0.02 mg/kg weight, 0.03 mg/kg weight, 0.04 mg/kg weight, 0.05 mg/kg weight, 0.06 mg/kg weight, 0.07 mg/kg weight, 0.08 mg/kg weight, 0.09 mg/kg weight, or 0.1 mg/kg weight. The effective dose for a particular individual can be varied (e.g., increased or decreased) over time, depending on the needs of the individual. [0143] In some embodiments, the Ang (1-7) peptide or MasR agonist is administered at an effective dose ranging from about 1-1,000 μg/kg/day (e.g., ranging from about 1-900 μg/kg/day, 1-800 μg/kg/day, 1-700 μg/kg/day, 1-600 μg/kg/day, 1-500 μg/kg/day, 1-400 μg/kg/day, 1-300 μg/kg/day, 1-200 μg/kg/day, 1-100 μg/kg/day, 1-90 μg/kg/day, 1-80 μg/kg/day, 1-70 μg/kg/day, 1-60 μg/kg/day, 1-50 μg/kg/day, 1-40 μg/kg/day, 1-30 μg/kg/day, 1-20 μg/kg/day, 1-10 μg/kg/day). In some embodiments, the Ang (1-7) peptide or MasR agonist is administered at an effective dose ranging from about 1-500 μg/kg/day. In some embodiments, the Ang (1-7) peptide or MasR agonist is administered at an effective dose ranging from about 1-100 μg/kg/day. In some embodiments, the angAng (1-7) peptide or MasR agonist is administered at an effective dose ranging from about 1-60 jig/kg/day. In some embodiments, the Ang (1-7) peptide or MasR agonist is administered at an effective dose selected from about 1, 2, 4, 6, 8, 10, 15, 20, 25, 30, 35, 40, 45, 50, 75, 100, 150, 200, 250, 300, 350, 400, 450, 500, 550, 600, 650, 700, 750, 800, 850, 900, 950, or 1.000 μg/kg/day.

Various embodiments of the presently disclosed subject matter can include differing dosing regimens. In some embodiments, the Ang (1-7) peptide or MasR agonist agonist is administered via continuous infusion. In some embodiments, the continuous infusion is intravenous. In other embodiments, the continuous infusion is subcutaneous. Alternatively or additionally, in some embodiments, the Ang (1-7) peptide or MasR agonist is administered bimonthly, monthly, twice monthly, triweekly, biweekly, weekly, twice weekly, thrice weekly, daily, twice daily, or on another clinically desirable dosing schedule. The dosing regimen for a single subject need not be at a fixed interval, but can be varied over time, depending on the needs of the subject.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

Without further description, it is believed that one of ordinary skill in the art can, using the preceding description and the following illustrative Examples, make and utilize the compounds of the presently disclosed subject matter and practice the methods of the presently disclosed subject matter. The following Examples therefore particularly point out embodiments of the presently disclosed subject matter and are not to be construed as limiting in any way the remainder of the disclosure.

### Materials and Methods for the Examples

Study Design. Discarded human plasma samples from COVID-19 positive and negative patients at the University of Virginia Medical Center (Charlottesville, Virginia, United States of America) were collected for cytokine, angiotensin peptide and growth factor analyses. The collection of biological specimens and de-identified patient information (no consent required) was approved by the University of Virginia Institutional Review Board (IRB-HSR #22231 and 200110).

Human Samples. Blood samples from 230 patients tested for SARS-CoV-2 by PCR between April and October 2020 were found using the University of Virginia Medical Center's electronic database. 166 of the 230 patients included in this study were SARS-CoV-2 positive and 64 were SARS-CoV-2 negative. For those patients with COVID-19, the earliest blood samples taken during emergency department visit or hospitalization at the University of Virginia were used for this analysis. Blood was collected into EDTA-containing vacutainers by a trained hospital phlebotomist. Blood was centrifuged at 1300×g for 10 minutes and after completion of biochemical testing, ordered by the clinician, the remaining plasma was stored at 4° C. for 48 hours before it was deemed “discarded” and released to the research laboratory. Plasma samples were aliquoted and stored at −80° C. until immediately prior to testing.

Patient Descriptors/Clinical Course. Demographics (age, gender, race), comorbidities, medication use, hospitalization status, lab results, and other clinical information were obtained by an honest broker from the electronic medical record (EMR; see Tables 1 and 4). Confidentiality was maintained by assigning each patient a unique identifier. Severity of COVID-19 illness was assessed through review of the EMR in several ways: first by inpatient admission vs outpatient care, second by the use of supplemental oxygen (none vs any supplemental oxygen, and supplemental oxygen delineated as low flow nasal canula vs mechanical ventilation or high-flow oxygen) and finally by mortality. Days from symptom onset were scored as per Lucas et al., 2020 based on the patient's determination or by the earliest reported symptom from the patient as recorded in the electronic medical record. All mean arterial blood pressure measurements from the day of sample collection were pulled from the electronic medical record and averaged prior to inclusion in all analyses.

Quantification of Ang II. Ang II was quantified in undiluted plasma using the Angiotensin II ELISA Kit (ALPCO, cat #74-ANGHU-E01) according to the manufacturer's instructions. Ang II measurements from 28 of the 230 aliquoted samples were excluded from the final analysis due to the samples undergoing multiple freeze-thaw cycles. Standard curves were prepared for each 96-well plate. The minimum and maximum detectable concentrations of Ang II was 4.6 pg/mL and 10,000 μg/mL respectively.

Quantification of Ang 1-7. Ang 1-7 was quantified in undiluted plasma using the Angiotensin 1-7 ELISA Kit (Novus Biologicals, cat #NBP2-69078). Manufacturer instructions were followed using 35 μL of sample per well. Ang 1-7 measurements from 1 of the 230 aliquoted samples was excluded from the final analysis due to insufficient sample volume. Standard curves were prepared for each 96-well plate. The minimum and maximum detectable concentrations of Ang 1-7 was 9.38 pg/mL and 1000 pg/mL respectively.

Cytokine quantification. Cytokine concentrations in plasma were measured using the MILLIPLEX® MAP Human Cytokine/Chemokine/Growth Factor Panel A (48 Plex; Millipore Sigma, St. Louis MO, Catalog Number HCYTA-60K-PX48) by the Flow Cytometry Facility of the University of Virginia. Cytokines detected were sCD40L, EGF, Eotaxin, FGF-2, Flt-3 ligand, Fractalkinc, G-CSF, GM-CSF, GROα, IFNα2, IFNγ, IL-1α, IL-1β, IL-Ira, IL-2, IL-3, IL-4, IL-5, IL-6, IL-7, IL-8, IL-9, IL-10, IL-12 (p40), IL-12 (p70), IL-13, IL-15, IL-17A, IL-17E/IL-25, IL-17F, IL-18, IL-22, IL-27, IP-10, MCP-1, MCP-3, M-CSF, MDC (CCL22), MIG, MIP-1α, MIP-1β, PDGF-AA, PDGF-AB/BB, TGFα, TNFα, TNFβ, and VEGF-A. RANTES was excluded.

D-dimer measurement. D-dimer levels determined by the clinical laboratory at the University of Virginia on the same day as samples were collected for this study were retrospectively pulled from the electronic medical record by an honest broker.

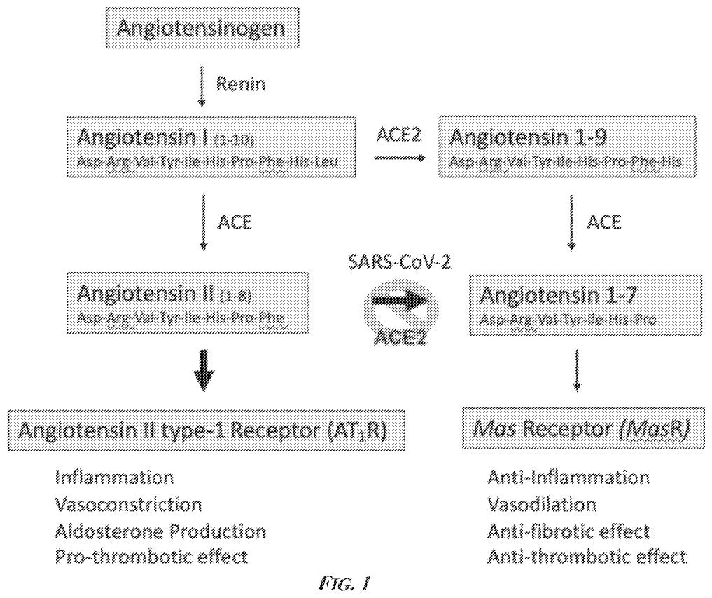

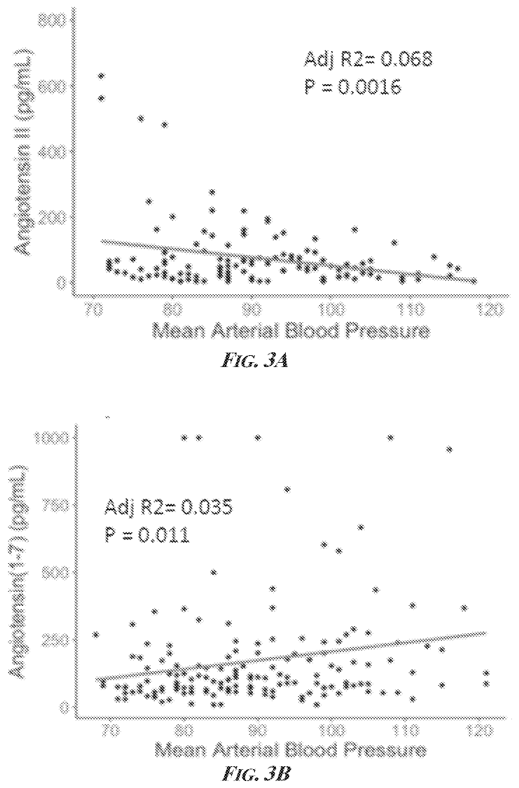

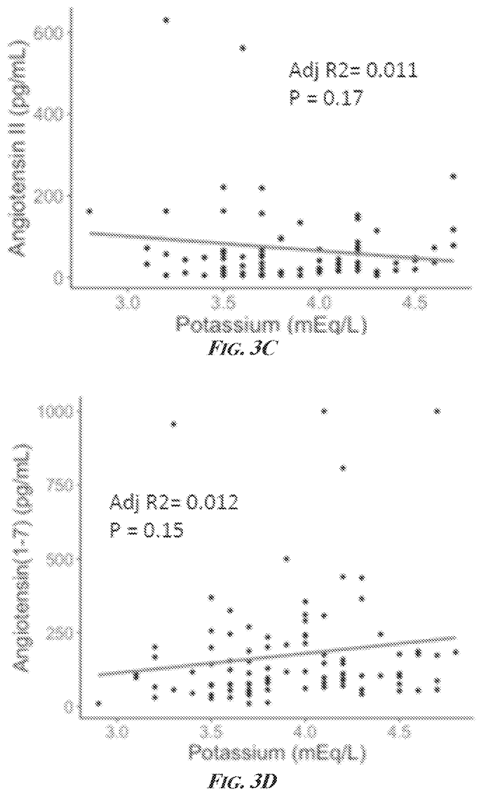

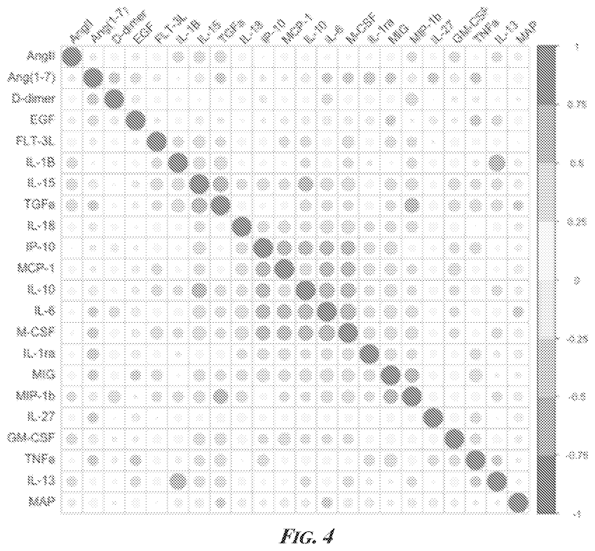

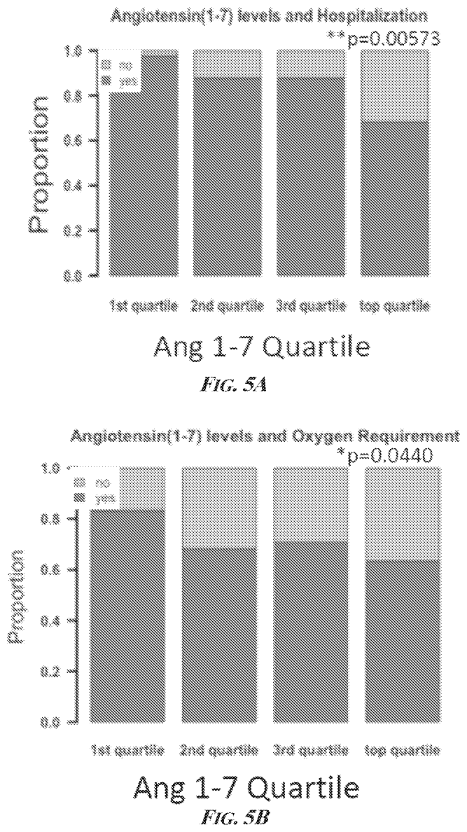

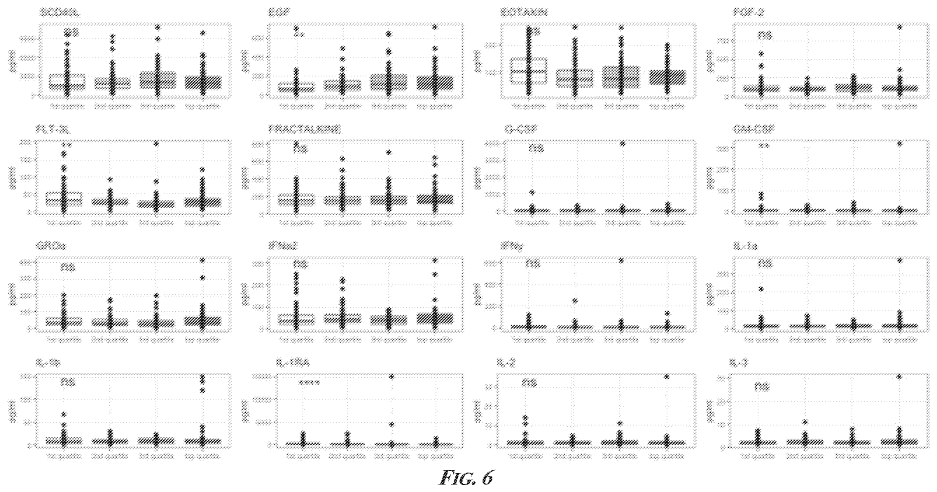

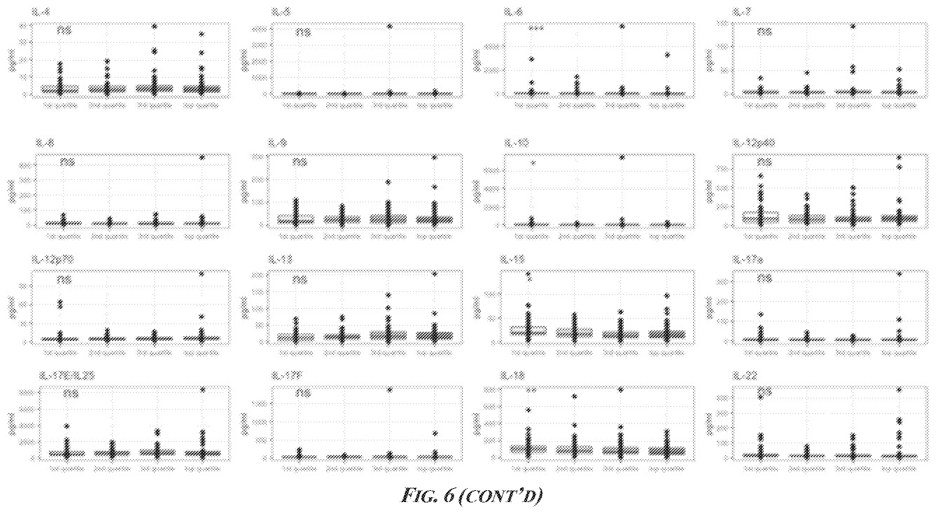

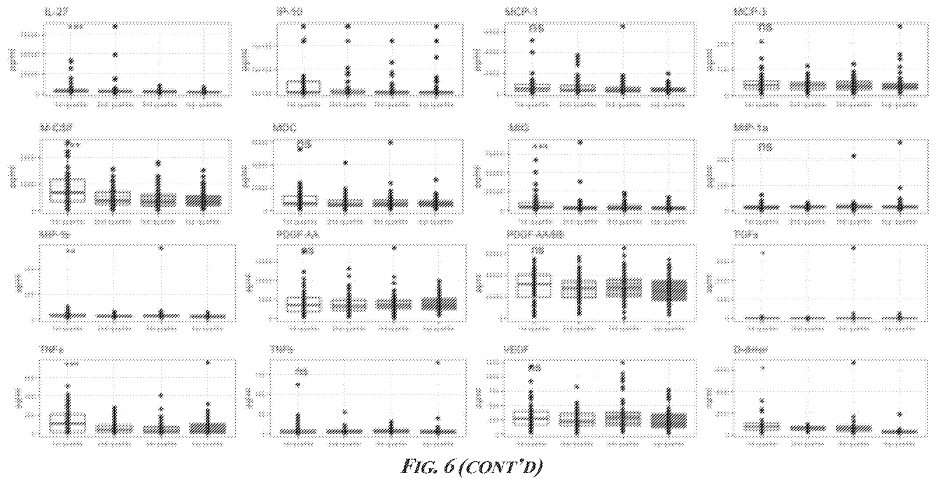

Statistical Methods. All statistical comparisons and graphs were made using R, version 4.0.3. Cases and controls were compared with respect to Ang II and Ang 1-7 using non-parametric Mann-Whitney U Tests. Key outcome indicators such as hospitalization, oxygen supplementation, ventilation and mortality were categorized as yes/no and also compared with respect to Ang II and Ang 1-7 using non-parametric Mann-Whitney U Tests. We estimated odds ratios (ORs) for the association of Ang peptides (and other independent variables) with adverse outcomes of COVID-19 using univariable logistic regression (FIG. 1). Independent variables identified in univariable models (p<0.10) were included in multivariable logistic regression. A p-value of <0.05 was considered statistically significant (Table 2). Patients were classified into 4 quartiles based on the cumulative distribution of Ang 1-7 levels, and the probabilities of hospitalization and oxygen supplementation using logistic regression (see FIGS. 3A-3D). Associations between individual inflammatory/coagulation markers and Ang 1-7 quartiles was detected using Kruskal-Wallis tests (p value<0.05 was considered statistically significant; see FIG. 6). Significant associations were further analyzed using a Spearman rank correlation to measure the degree of the association between inflammatory/coagulation markers and angiotensin peptides inputted as individual continuous variables. Results are summarized as showing a weak (0-0.25), medium (0.25-0.5) or strong (>0.5) association (FIG. 4).

### Example 1

### Patient Characteristics

Clinical and demographic characteristics of COVID-19 patients and control subjects are displayed in Table 1. Their age was 57.1±17.7 years (14.0-104.0 min-max) and 55% were males. A significantly lower percentage of COVID-19 patients compared to control subjects were Caucasian and a significantly greater percentage were Hispanic. Significant differences were noted in BMI, cancer and diabetes diagnoses between the SARS-CoV-2 positive and negative groups. A majority of the patients had at least one comorbidity. Among the 166 COVID-19 patients, 142 were hospitalized, 118 required oxygen supplementation, 61 required ventilation and 25 died of the disease. Demographics and characteristics by patient outcomes are shown in Table 4. On average, the samples used for this study were collected 10.5 days after symptom onset. Seventy-three COVID-19 patients (44.0%) received steroids and 39 (23.8%) received Remdesivir during the course of their infection. Of note, while ACE inhibitor (ACEi) and angiotensin receptor blocker (ARB) use were included in this analysis, these prescribed medications represent long-term use and were not administered during the acute hospitalization.

### Example 2

### Reduced an& 1-7 Levels Associated with Severe Disease

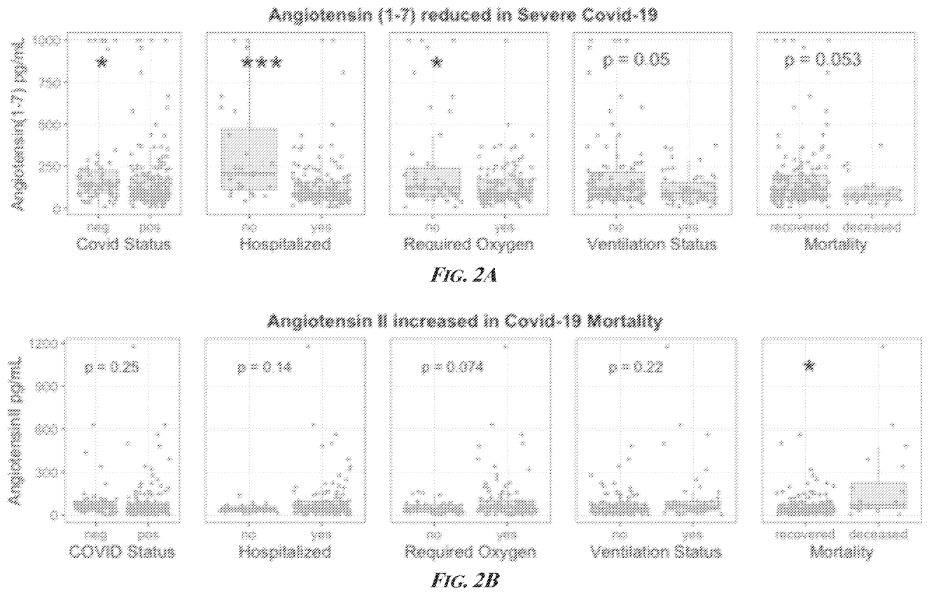

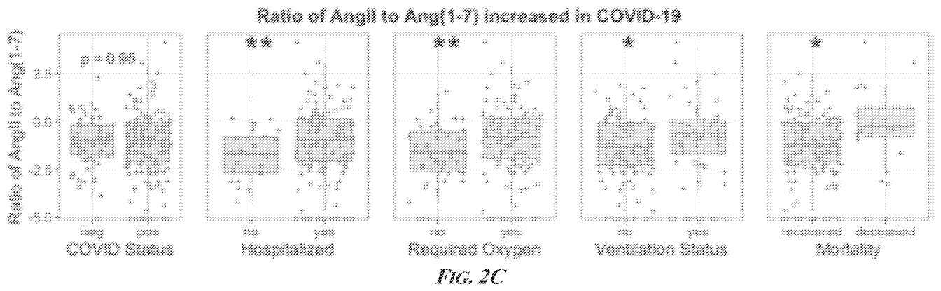

Ang 1-7 levels were reduced in COVID-19 patients compared to control subjects and decreasing levels of this peptide correlated with disease severity (FIG. 2A). We observed a difference in plasma Ang 1-7 between COVID-19 patients and controls (Wilcoxon Mann-Whitney p=0.015). In the COVID-19 patient group, the median (IQR) was 104 (66-186) pg/mL and in the control group the median (IQR) was 143 (94-229) pg/mL. We also observed reduced Ang 1-7 in patients requiring hospitalization (p=0.00016), oxygen supplementation (p=0.0379), ventilation (p=0.05) and among those who died of the disease (p=0.053). The median (IQR) in the hospitalized group was 90 (60-159) pg/mL vs 206 (113-474) pg/mL in the group that did not require hospitalization. The median (IQR) Ang 1-7 of those that required oxygen was 92 (60 to 172) pg/mL versus 122 (76 to 242) pg/mL among those who did not require oxygen. Of the ventilated group the median (IQR) was 92 (57-154) pg/mL versus 114 (70-217) pg/mL in the group that did not require ventilation. Finally, the median (IQR) Ang 1-7 of those who died of COVID-19 was 79 (54-122) pg/mL versus 110 (70-198) pg/mL in the group that recovered.

It is important to note that these measurements reflect circulating levels of Ang II and Ang 1-7 which are one-step removed from the local tissue levels where SARS-CoV-2 directly interacts with the ACE-2 receptor. Given the extreme heterogeneity of COVID-19 in terms of patients impacted and pathophysiologic responses, any significant difference detected at the systemic level may reflect a larger difference at the tissue level where SARS-CoV-2 inflicts damage (Henry et al., 2020; Wiese et al., 2020).

We observed an increasing trend of Ang II in more severe outcomes of COVID-19, however mortality was the only endpoint for which the difference was statistically significant (Wilcoxon Mann-Whitney p=0.0142; FIG. 2B). The median (IQR) Ang II in the group that died from COVID-19 was 69 (47-225) pg/mL and in the group that recovered the median (IQR) was 45 (19-76) pg/mL. Ang II binds to AT₁Rs on renal juxtaglomerular cells, serving as a well-known negative feedback regulator of juxtaglomerular cell renin release and, thus, plasma renin activity. Therefore, it might be predicted that reduced ACE2 activity in COVID-19 would have a relatively larger impact on Ang 1-7 levels than on Ang II, which would be expected to rapidly decline as a result of the AT₁R short-loop negative feedback mechanism (Friis et al., 2013). It is also important to note that the ratio of Ang II:Ang 1-7 is significantly increased in more severe COVID-19 cases (FIG. 2C).

To further explore RAS dysregulation in this cohort we examined blood pressure and potassium which are both impacted by Ang II levels. Ang II activates AT₁Rs which stimulate adrenal aldosterone production (Rieder et al., 2021). Aldosterone acts at the renal cortical collecting duct to promote sodium reabsorption and extracellular fluid volume expansion and increase potassium excretion (Carey, 2015). Thus, Ang II causes a rise in blood pressure (BP), whereas Ang 1-7 via MasR opposes this mechanism leading to natriuresis and reduction in BP (Carey, 2015). Interestingly, in patients with COVID-19, the opposite relationship of that anticipated between Ang II, Ang 1-7 and BP was observed (FIGS. 3A and 3B). Ang II was negatively correlated, while Ang 1-7 was positively correlated with BP in COVID-19. This finding is consistent with an association between reduced ACE2 activity (reflected by increasing Ang II and reduced Ang 1-7) and increased inflammation leading to lower BP in more severe cases of COVID-19. Of note, BP negatively correlated with proinflammatory cytokines (FIG. 4). However, it is uncertain whether this was a direct cause of inflammation or was confounded by the high rates of sedation and positive pressure ventilation in more severe manifestations of disease. We did not observe a significant association between Ang peptides and potassium in this cohort. However, we were unable to control for potassium repletion and the impact of acute kidney injury, which occurred in over 30% of patients, on circulating potassium levels (FIGS. 3C and 3D).

### Example 3

### Ang 1-7 Predicts Need for Hospitalization and Oxygen Supplementation in COVID-19

We fit logistic regression models for hospitalization, oxygen supplementation, ventilation and mortality separately with predictors including Ang II, Ang 1-7, age, sex, race, BMI, ACEi/ARB use and presence of any comorbidity, categorized as yes/no (Table 2). In univariable analysis, Ang 1-7, age and the presence of any comorbidity were associated with need for hospitalization. All of these, in addition to Ang II and ACE inhibitor (ACEi)/AT₁R blocker (ARB) use were associated with need for oxygen supplementation. Table 2 also illustrates multivariable logistic regression analyses using significant variables selected during univariable analyses. Every 10 μg/mL unit increase of plasma Ang 1-7 was associated with a 3% reduction in odds of hospitalization (AOR 0.97, CI 0.95 to 0.99) and a 3% reduction in odds of requiring oxygen supplementation (AOR 0.97, CI 0.95 to 0.99) and/or ventilation (AOR 0.97, (CI 0.94 to 0.99). No significant association was noted on multivariable analysis between Ang 1-7 levels and mortality (Table 2). In contrast, as we might expect, every 10 μg/mL increase in Ang II was associated with a 4% increase in odds of mortality on multivariable analysis (AOR 1.04, CI 1.01 to 1.08).

Since many of the patients included in this analysis contracted COVID-19 prior to the establishment of standard treatments for the disease, fewer than half of these patients received immunomodulatory and antiviral therapy. Of those who did, the majority received a single dose prior to blood sample collection. Regarding the influence of immunomodulatory therapy on RAS peptide levels, median Ang 1-7 concentrations in patients treated with corticosteroids (compared with no corticosteroids) were 110 (interquartile range [IQR], 72-187) pg/ml and 91 (IQR, 60-178) pg/ml, respectively (P=0.534). Median Ang II concentrations in those treated with corticosteroids (compared to no corticosteroids) were 58 (IQR, 25-115) pg/mL and 44 (IQR, 18-69) pg/ml, respectively (P=0.072). Antiviral treatment did not have a statistically significant correlation with RAS metabolite concentrations (data not shown).

### Example 4

### An 1-7. Inflammation and Coagulation

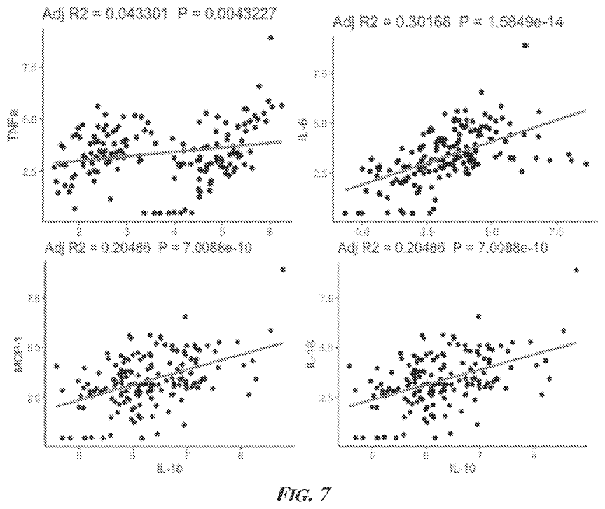

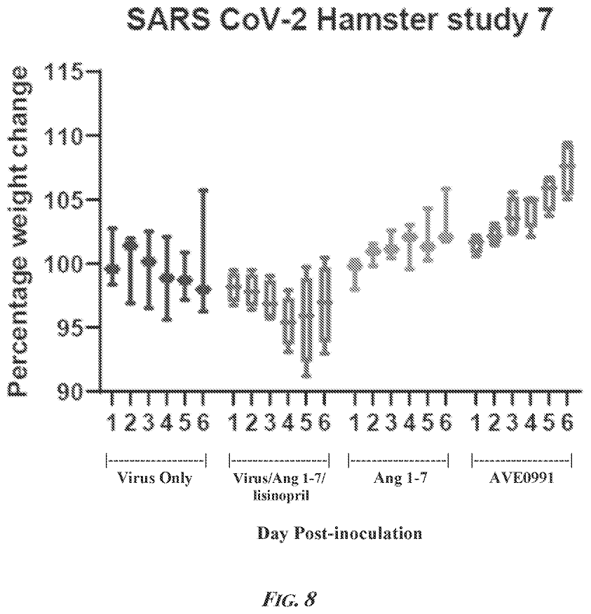

Ang 1-7 has anti-inflammatory and anti-thrombotic effect via activation of the MasR (Fraga-Silva et al., 2011, Fang et al., 2013). This peptide inhibits pro-inflammatory cytokines IL-6, TNF-α, IL-1beta and MCP-1 through the NF-kB, JNK and ERK 1/2 pathways (El-Hashim et al., 2012; Simões e Silva et al., 2013). In order to investigate the impact of Ang 1-7 on the inflammatory response to SARS-CoV-2, we grouped patients into Ang 1-7 quartiles where a significantly greater proportion of the lowest versus highest groupings required hospitalization and oxygen supplementation (FIGS. 5A and 5B; Table 3). We then evaluated each cytokine, growth factor, and D-dimer individually for significant differences across Ang 1-7 quartiles (see FIG. 6). Factors identified as having significant associations with Ang 1-7 were incorporated into a correlation matrix to determine the strength and direction of the association (see FIG. 4). As expected, Ang II was positively correlated with most pro-inflammatory cytokines while Ang 1-7 was negatively correlated with both cytokines and D-dimer levels. A medium strength association was detected between D-dimer, pro-inflammatory cytokines IL-6 and TNFa as well as MIG and M-CSF which are involved in monocyte signaling. This finding supports our hypothesis that increasing levels of Ang 1-7 is protective in COVID-19 via inhibition of thrombosis and inflammation (Henry et al., 2020; McFadyen et al., 2020). Interestingly anti-inflammatory cytokine IL-10 was also negatively associated with Ang 1-7. On further exploration, this increase in IL-10 in lower Ang 1-7 groups was associated with immune activation of pro-inflammatory cytokines, and could be a compensatory response (FIG. 7; see also Couper et al., 2008).

### Discussion of the Examples

Here we have shown that reduced Ang 1-7 is associated with hospitalization, oxygen supplementation and ventilation in COVID-19, particularly when adjusted for age, race, use of ACEi/ARBs and comorbidity status. The most significant difference in Ang 1-7 levels was detected between relatively asymptomatic cases and patients requiring some degree of medical intervention.

Our findings here are in contradistinction to a recent comprehensive meta-analysis which found over-activation, rather than depression, of the protective arm of the RAS in COVID-19 (Pucci et al., 2021). This meta-analysis does note significant differences among the results of the included studies but reports that on average Ang 1-7 levels are approximately 10 times higher in COVID-19 patients versus controls (Pucci et al., 2021). Several explanations could account for discrepancy in these findings. First, this meta-analysis combined a range of studies that employed different sample collection and processing methods. The largest included study employed equilibrium analysis which does not utilize protease inhibitors and reflects the ongoing activity of RAS proteases (Reindl-Schwaighofer et al., 2021; van Lier et al., 2021). Given that ACE2 is cleaved from the cell surface and circulating levels are increased in COVID-19, equilibrium analysis does not provide a reliable indication of RAS metabolite levels in the body tissues as Ang 1-7 production continues after serum collection. Other studies allowed blood samples to clot at room temperature and reported RAS peptide levels at surprisingly low concentrations suggesting that ongoing peptide metabolism confounded the findings (Burns et al., 2021; Files et al., 2021; Osman et al., 2021). Second, most studies investigating RAS dysregulation in COVID-19 are small and underpowered with the largest study including only 126 patients, of which only 32 were severely ill (Reindl-Schwaighofer et al., 2021). Other studies include few to no severely ill COVID-19 patients (Burns et al., 2021; Files et al., 2021; Osman et al., 2021; van Lier et al., 2021).

Our study agrees with findings of Henry et al. who found Ang 1-7 levels to be significantly lower in patients with COVID-19 compared to controls and in those admitted to the ICU versus those who did not require intensive care (Henry et al., 2021). Similarly, Wu et al. and Liu et al. reported significant elevations in Ang II levels in critically ill COVID-19 cases compared to controls/mild cases (Liu et al., 2020; Wu et al., 2020).

A strength of the presently disclosed subject matter is the inclusion of a large COVID-19 patient cohort with disease phenotypes ranging from asymptomatic to critically ill. This large and diverse cohort has allowed for the identification of RAS dysregulation in COVID-19 that was consistent across outcomes and significant upon adjusted multivariable analysis. Given the low endogenous concentration of the RAS peptides and the considerable heterogeneity seen in COVID-19 patients, a large patient cohort with the power to detect small changes in the RAS is critical to successful determination of the impact of SARS-CoV-2 on this tightly regulated system (Kohara et al., 1991; Kintscher et al., 2020). Given the sample size of 166 COVID positive patients, we were also able to control for the impact of race, age, comorbidities, and long-term ACEi/ARB use in multivariate analysis. Dexamethasone, which is now standard of care in severe COVID-19 upregulates ACE and ACE2 and decreases morbidity and mortality (Sinha et al., 2020). To ensure immunomodulatory therapy did not confound the current analysis, we compared the initial RAS metabolite levels in those who received treatment versus those who did not and found no significant difference. Further studies at various time points before, during and after treatment are needed to determine if dexamethasone provides therapeutic benefit via upregulation of ACE2 and subsequent alterations in RAS metabolite levels.

Another strength is the pairing Ang peptide with cytokine and D-dimer levels which demonstrated significant associations consistent with prior research about the role of the ACE2/Ang 1-7/MasR pathway in modulating inflammation and coagulation (Fang et al., 2013; Simões e Silva et al., 2013). To our knowledge, the presently disclosed subject matter represents the first to examine the association of RAS peptides and cytokine/D-dimer levels in COVID-19. Association of reduced Ang 1-7 with increased inflammation/thrombosis strengthens the level of evidence that disruptions in this counter-regulatory pathway are involved in the pathogenesis of COVID-19 (Angeli et al., 2021). The mechanism(s) by which Ang 1-7 protects against severe disease is/are unknown, though some clues can be taken from the literature and from correlations in this cohort with inflammatory markers and D-dimer levels. Many studies have shown that the RAS, composed of the ACE/Ang II/AT₁R axis and the counterregulatory ACE2/Ang 1-7/MasR pathway, plays a relevant role in the pathogenesis of inflammatory diseases (Simbōes a Silva et al., 2013). Ang II is known to activate signaling pathways related to tissue injury, inflammation and fibrosis including activation of the transcription factor NFkB, recruitment of inflammatory cells, adhesion of monocytes and neutrophils to endothelial and mesangial cells and synthesis and release of cytokines and chemokines including IL-1β (da Silveira et al., 2010). Evidence suggests that Ang 1-7 opposes these actions, as the heptapeptide has been shown to down-regulate mRNA levels of pro-inflammatory cytokines IL-6 and TNFa, negatively modulate leukocyte migration and decrease the frequency of M1 inflammatory macrophage phenotypes (da Silveira et al., 2010; de Carvalho Santuchi et al., 2019). SARS-CoV-2 appears to down-regulate the expression of ACE-2 on peripheral blood monocytes which show an activated phenotype in COVID-19 evidenced by morphology and IL-6, IL-10 and TNFa production (Pence, 2020; Osman et al., 2021; Zhang et al., 2021). Monocyte activation appears to associate with disease severity and macrophage accumulation has been noted in COVID-19 patients on autopsy along with diffuse alveolar damage, pulmonary edema, fibrin deposition in the alveolar space and diffuse microvascular thrombi (Pence, 2020; Zipeto et al., 2020). It is interesting to note that Ang II was positively correlated with IL-1beta and GM-CSF in this cohort while Ang 1-7 was negatively correlated with IL-6, TNFa and M-CSF (FIG. 4). While further research is needed to define the mechanism by which RAS dysfunction impacts the course of COVID-19, it is possible that reduced Ang 1-7 levels favor a pro-inflammatory activation of macrophages in severe disease. Further investigation should also determine if RAS dysfunction is related to the pulmonary fibrosis and long-term sequelae of severe COVID-19 (George et al., 2020).

Ang 1-7 levels are also relevant to the risk of thrombosis in COVID-19 through a relatively direct mechanism. Ang 1-7 normally acts on the Mas and ATE receptors to increase the production of nitric oxide and prostacyclin which in turn contribute to vasodilation, reduced platelet spreading and collagen activation (Fang et al., 2013). Loss of this protective activity in COVID-19 likely contributes to the diffuse microvascular thrombi seen in many patients. This is supported by our finding that Ang 1-7 is negatively correlated with D-dimer levels in COVID-19 patients, suggesting reduced thrombi formation and break-down amongst patients with higher Ang 1-7 levels (FIG. 4).

Summarily, the presently disclosed subject matter pertains in some embodiments to the identification of disruptions in the protective ACE2/Ang 1-7/Mas receptor(MasR) arm of the RAS in patients treated for COVID-19 of varying severities. The association of the classical ACE/Ang II/AT₁R and the counter-regulatory ACE2/Ang 1-7/MasR with severity of COVID-19 and with markers of inflammation and coagulation was investigated. Angiotensin [Ang] II and Ang 1-7 were measured in plasma by ELISA in 230 patients, 166 who were COVID-19-positive. Ang 1-7 was repressed in COVID-19 compared to SARS-CoV-2 negative controls. Furthermore, multivariable logistic regression analyses demonstrated that every 10 μg/mL increase of plasma Ang 1-7 was associated with a 3% reduction in odds of hospitalization (AOR 0.97, CI 0.95 to 0.99) and a 3% reduction in odds of requiring oxygen supplementation (AOR 0.97, CI 0.95 to 0.99) and/or ventilation (AOR 0.97, CI 0.94 to 0.99). Ang 1-7 was inversely associated with pro-inflammatory cytokines and D-dimer in this patient cohort suggesting that reduced activity in this protective counter-regulatory arm of the RAS contributes to the hyper-immune response and diffuse coagulation activation documented in COVID-19.

These data suggest a role for pathologic modulation of ACE2 in COVID-19 leading to repressed Ang 1-7 levels and inversely to a rise in pro-inflammatory and thrombotic markers. Among the findings disclosed herein are the following:
High levels of the peptide Ang II were associated with mortality from COVID-19.
Low levels of the peptide Ang 1-7 were associated with COVID-19 infection and severe outcome indicators including hospitalization, oxygen supplementation and ventilation.
Increased ratios of Ang II:Ang(1-7) were associated with severe outcome indicators including hospitalization, oxygen supplementation, ventilation and mortality.
Ang II levels were positively correlated with D-dimer and pro-inflammatory cytokines, including IL-1B.
Ang 1-7 levels were negatively correlated with D-dimer and pro-inflammatory cytokines, including IL-6. TNFα, and M-CSF.

Thus, in some embodiments, the presently disclosed subject matter addresses at least two objects: (1) it offers support for the role of ACE2 disruption in the pathogenesis and severity of COVID-19; and (2) it reports the discovery of a correlation between repressed Ang 1-7 and increasing inflammation and thrombosis in COVID-19 of infections with coronaviruses including but not limited to SARS-CoV-2.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Altschul et al. (1990a) Basic local alignment search tool. J Mol Biol 215:403-410.
Altschul et al. (1990b) Protein database searches for multiple alignments. Proc Natl Acad Sci USA 87:14:5509-13.
Altschul et al. (1997) Gapped BLAST and PSI-BLAST: a new generation of protein database search programs. Nucleic Acids Res 25:3389-3402.
Angeli et al. (2021) The pivotal link between ACE2 deficiency and SARS-CoV-2 infection: One year later. Eur J Intern Med 93:28-34.
Behzad et al. (2020) Extrapulmonary manifestations of COVID-19; radiologic and clinical overview. Clin Imaging 66:35-41.
Burns et al. (2021) Sustained dysregulation of the plasma renin-angiotensin system in acute COVID-19. Research Square 2021 Preprint. DOI: https://doi.org/10.21203/rs.3.rs-125380/vl.
Carey (2015) The intrarenal renin-angiotensin system in hypertension. Adv Chronic Kidney Dis 22:204-10.
Chen et al. (2020) Individual variation of the SARS-CoV-2 receptor ACE2 gene expression and regulation. Aging Cell 19:e13168.
Couper et al. (2008) IL-10: the master regulator of immunity to infection. J Immunol 180:5771-7.
da Silveira et al. (2010) Anti-inflammatory effects of the activation of the angiotensin-(1-7) receptor. MAS, in experimental models of arthritis. J Immunol 185:5569-5576.
de Carvalho Santuchi et al. (2019) Angiotensin-(1-7) and alamandine promote anti-inflammatory response in macrophages In Vitro and In Vivo. Mediators Inflamm 2019:2401081.
Devereux et al. (1984) A comprehensive set of sequence analysis programs for the VAX. Nucleic Acids Research 12:387-395.
El-Hashim et al. (2012) Angiotensin-(1-7) inhibits allergic inflammation, via the MASI receptor, through suppression of ERK1/2- and NF-κB-dependent pathways. Br J Pharmacol 166:1964-1976.
Fang et al. (2013) Angiotensin 1-7 and Mas decrease thrombosis in Bdkrb2−/− mice by increasing NO and prostacyclin to reduce platelet spreading and glycoprotein VI activation. Blood 121:3023-32.
Ferrario (2011) ACE2: more of Ang-(1-7) or less Ang II?. Curr Opin Nephrol Hypertens 20:1-6.
Files et al. (2021) A pilot study to assess the circulating renin-angiotensin system in COVID-19 acute respiratory failure. Am J Physiol Lung Cell Mol Physiol 321(1):L213-L218.
Fraga-Silva et al. (2011) An orally active formulation of angiotensin-(1-7) produces an antithrombotic effect. Clinics (Sao Paulo) 66:837-841.
Friis et al. (2013) Regulation of renin secretion by renal juxtaglomerular cells. Pflugers Arch 465:25-37.
Genaro, ed. (1985) Remington's Pharmaceutical Sciences, Mack Publishing Co., Easton, Pennsylvania, United States of America.
George et al. (2020) Pulmonary fibrosis and COVID-19: the potential role for antifibrotic therapy. Lancet Respir Med 8:807-815.
Gheblawi et al. (2020) Angiotensin-converting enzyme 2: SARS-CoV-2 Receptor and regulator of the renin-angiotensin system: celebrating the 20th anniversary of the discovery of ACE2. Circ Res 126(10):1456-1474.
Green et al. (2008) A combinatorial polymer library approach yields insight into nonviral gene delivery. Ace Chem Res 41(6):749-759.
Gross & Mienhofer (eds.) (1981) The Peptides. Vol. 3, Academic Press, New York, New York, United States of America. pages 3-88.
Henry et al. (2020) Hyperinflammation and derangement of renin-angiotensin-aldosterone system in COVID-19: A novel hypothesis for clinically suspected hypercoagulopathy and microvascular immunothrombosis. Clin Chim Acta 507:167-173.
Henry et al. (2021) Coronavirus disease 2019 is associated with low circulating plasma levels of angiotensin 1 and angiotensin 1,7. J Med Virol 93(2):678-680.
Iba et al. (2020) The unique characteristics of COVID-19 coagulopathy. Crit Care 24(1):360.
Imai et al. (2005) Angiotensin-converting enzyme 2 protects from severe acute lung failure. Nature 436(7047):112-116.
Karlin & Altschul (1990) Methods for assessing the statistical significance of molecular sequence features by using general scoring schemes. Proc Nat Acad Sci USA 87:2264-2268.
Karlin & Altschul (1993) Applications and statistics for multiple high-scoring segments in molecular sequences. Proc Natl Acad Sci USA 90:5873-5877.
Khan et al. (2017) A pilot clinical trial of recombinant human angiotensin-converting enzyme 2 in acute respiratory distress syndrome. Crit Care 21(1):234.
Kintschcr et al. (2020) Plasma angiotensin peptide profiling and ACE (Angiotensin-Converting Enzyme)-2 activity in COVID-19 patients treated with pharmacological blockers of the renin-angiotensin system. Hypertension 76:e34-e36.
Kohara et al. (1991) Reassessment of plasma angiotensins measurement: effects of protease inhibitors and sample handling procedures. Peptides 12:1135-1141.
Kuba et al. (2005) A crucial role of angiotensin converting enzyme 2 (ACE2) in SARS coronavirus-induced lung injury. Nat Med 11(8):875-879.
Liu et al. (2020) Clinical and biochemical indexes from 2019-nCoV infected patients linked to viral loads and lung injury. Sci China Life Sci 63:364-374.
Lucas et al. (2020) Longitudinal analyses reveal immunological misfiring in severe COVID-19. Nature 584:463-469.
McFadyen et al. (2020) The emerging threat of (micro)thrombosis in COVID-19 and its therapeutic implications. Circ Res 127:571-587.
Osman et al. (2021) Expression of ACE2 receptor, soluble ACE2, angiotensin I, angiotensin II and angiotensin(1-7), is modulated in COVID-19 patients. Frontier Microbiology 12:625732.
PCT International Patent Application Publication No. WO 2021/174107.
Pence (2020) Severe COVID-19 and aging: are monocytes the key? Geroscience 42(4):1051-1061.
Ponti et al. (2020) Biomarkers associated with COVID-19 disease progression. Crit Rev Clin Lab Sci 57(6):389-399.
Pucci et al. (2021) Quantifying Renin-Angiotensin-System Alterations in COVID-19. Cells 10(10):2755.
Ramos et al. (2021) ACE2 Down-Regulation May Act as a Transient Molecular Disease Causing RAAS Dysregulation and Tissue Damage in the Microcirculatory Environment Among COVID-19 Patients. Am J Pathol 191(7):1154-1164.
Reindl-Schwaighofer et al. (2021) ACE2 Elevation in Severe COVID-19. Am J Respir Crit Care Med 203(9):1191-1196.
Rieder et al. (2021) Serum ACE2, angiotensin II, and aldosterone levels are unchanged in patients with COVID-19. Am J Hypertens 34:278-281.
Simões a Silva et al. (2013) ACE2, angiotensin-(1-7) and Mas receptor axis in inflammation and fibrosis. Br J Pharmacol 169:477-492.
Sinha et al. (2020) In vitro and in vivo identification of clinically approved drugs that modify ACE2 expression. Mol Syst Biol 16(7):e9628.
Uhal et al. (2012) Angiotensin signalling in pulmonary fibrosis. Int J Biochem Cell Biol 44(3):465-468.
U.S. Patent Application Publication No. 2018/0289779.
van Lier et al. (2021) Increased blood angiotensin converting enzyme 2 activity in critically ill COVID-19 patients. ERJ Open Res 7(1):00848-2020.
Vogels et al. (2020) Analytical sensitivity and efficiency comparisons of SARS-CoV-2 RT-qPCR primer-probe sets. Nat Microbiol 5(10):1299-1305.
Wiese et al. (2020) Molecules in pathogenesis: angiotensin converting enzyme 2 (ACE2). J Clin Pathol 74(5):285-290.
World Health Organization (2021) Weekly Operational Update on COVID-19, 6 Apr. 2021. Accessed Apr. 8, 2021.
Wösten-van Asperen et al. (2011) Acute respiratory distress syndrome leads to reduced ratio of ACE/ACE2 activities and is prevented by angiotensin-(1-7) or an angiotensin II receptor antagonist. J Pathol 225(4):618-627.
Wösten-van Asperen et al. (2013) Imbalance between pulmonary angiotensin-converting enzyme and angiotensin-converting enzyme 2 activity in acute respiratory distress syndrome. Pediatr Crit Care Med 14(9):e438-441.
Wu et al. (2020) Elevation of plasma angiotensin II level is a potential pathogenesis for the critically ill COVID-19 patients. Crit Care 24:290.
Zhang et al. (2021) Frontline Science: COVID-19 infection induces readily detectable morphologic and inflammation-related phenotypic changes in peripheral blood monocytes. J Leukoc Biol 109:13-22.
Zipeto et al. (2020) ACE2/ADAM17/TMPRSS2 interplay may be the main risk factor for COVID-19. Front Immunol 11:576745.

It will be understood that various details of the presently disclosed subject matter can be changed without departing from the scope of the presently disclosed subject matter. Furthermore, the foregoing description is for the purpose of illustration only, and not for the purpose of limitation.

TABLE 1
Patient Demographics and Clinical Characteristics Stratified by COVID-19 (n = 230)

| | Number (%) | | | | |
|---|---|---|---|---|---|
| | COVID⁻ | | COVID⁺ | | |
| Demographics | (n = 64) | | (n = 166) | | P value* |
| Age (mean, SD) | 51.56 | (20.24) | 57.07 | (17.66) | <0.05 |
| Female | 32 | (50.0) | 74 | (44.6) | 0.554 |
| Male | 32 | (50.0) | 92 | (55.4) | |
| White or Caucasian | 41 | (64.1) | 60 | (36.1) | <0.001 |
| Asian | 1 | (1.6) | 5 | (3.0) | |
| Other | 7 | (10.9) | 61 | (36.7) | |
| African American | 15 | (23.4) | 40 | (24.1) | |
| Hispanic | 1 | (1.6) | 67 | (40.4) | <0.001 |
| Comorbidities | | | | | |
| Cardiac dysfunction | 14 | (21.9) | 28 | (16.9) | 0.490 |
| Chronic Kidney Disease | 11 | (17.2) | 28 | (16.9) | 1.000 |
| Lung Disease | 15 | (23.4) | 27 | (16.3) | 0.284 |
| Liver Disease | 3 | (4.7) | 3 | (1.8) | 0.443 |
| Stroke | 8 | (12.5) | 13 | (7.8) | 0.397 |
| Immunosuppression | 9 | (14.1) | 11 | (6.6) | 0.125 |
| Cancer | 15 | (23.4) | 14 | (8.4) | <0.05 |
| Diabetes | 17 | (26.6) | 67 | (40.4) | <0.05 |
| Body Mass Index | | | | | |
| <30 (not obese) | 38 | (61.3) | 63 | (43.8) | <0.05 |
| >30 (obese) | 24 | (38.7) | 81 | (56.2) | |
| Medications | | | | | |
| ACE inhibitor prior to admission | 12 | (18.8) | 41 | (24.7) | 0.067 |
| Angiotensin receptor blocker prior to admission | 8 | (12.5) | 16 | (9.6) | |

| COVID-19 Clinical Indicators and Treatments | | | |
|---|---|---|---|
| Received Steroids (%) | N.A. | 73 | (44.0) |
| Received Remdesivir (%) | N.A. | 39 | (23.8) |
| D-dimer³ (Mean, SD) | N.A. | 795.06 | (1,006.82) |
| Mean Arterial Pressure (Day of Sample) | | 89.42 | (12.25) |
| (Mean, SD) | | | |
| Days from Symptom Onset to Sample | N.A | 10.50 | (11.03) |
| (Mean, SD) | | | |
| Hospitalization Status (%) | | | |
| Admitted | N.A. | 142 | (85.5) |
| Not Admitted | N.A. | 24 | (14.5) |
| Oxygen Requirement (%) | | | |
| None | N.A. | 48 | (28.9) |
| Low-flow oxygen | N.A. | 54 | (32.5) |
| High-flow oxygen | N.A. | 3 | (1.8) |
| Mechanical ventilation | N.A. | 61 | (36.7) |
| Mortality (%) | | | |
| Deceased | N.A. | 25 | (15.1) |
| Recovered | N.A. | 141 | (84.9) |

*P-values from chi-square analysis and fisher's exact test for categorical variables and from t-test for continuous variables

TABLE 2
Predictors of Hospitalization and Severe Respiratory Illness in COVID-19

| | Unadjusted | | Adjusted | |
|---|---|---|---|---|
| Hospitalization | OR (95% CI)* | P value* | AOR (95% CI) | P value |
| Ang II (per 10 pg/mL) | 1.10 (1.01 to 1.24) | 0.0708 | 1.08 (0.99 to 1.24) | 0.195 |
| Ang 1-7 (per 10 pg/mL) | 0.96 (0.94 to 0.98) | 0.000274** | 0.97 (0.95 to 0.99) | 0.00989** |
| Age (per 10 years) | 1.74 (1.32 to 2.37) | 0.000164*** | 1.52 (1.06 to 2.27) | 0.0309* |
| Sex (male) | 1.40 (0.74 to 2.68) | 0.302 | — | — |
| Race (ref = White) | | | | |
| African American | 0.29 (0.07 to 0.98) | 0.0544 | 0.15 (0.02 to 0.71) | 0.0234* |
| Asian | 0.12 (0.01 to 0.98) | 0.0333 | 0.30 (0.02 to 3.85) | 0.337 |
| Other | 0.36 (0.10 to 1.16) | 0.105 | 0.73 (0.15 to 3.04) | 0.666 |
| Ethnicity (Hispanic) | 0.94 (0.39 to 2.32) | 0.888 | — | — |
| BMI (>30 obese) | 1.26 (0.41 to 3.89) | 0.678 | — | — |
| ACE inhibitor | 4.38 (1.20 to 28.29) | 0.0538 | 2.76 (0.53 to 22.18) | 0.268 |
| Angiotensin receptor blocker | 1.57 (0.40 to 10.52) | 0.569 | 0.74 (0.12 to 6.38) | 0.754 |
| Any Comorbidity | 4.76 (1.91 to 13.03) | 0.00124** | 2.33 (0.74 to 7.74) | 0.153 |
| Oxygen requirement | | | | |
| Ang II (per 10 pg/mL) | 1.07 (1.02 to 1.16) | 0.0327* | 1.07 (1.01 to 1.17) | 0.0903 |
| Ang 1-7 (per 10 pg/mL) | 0.97 (0.95 to 0.99) | 0.00332** | 0.97 (0.95 to 0.99) | 0.0144* |
| Age (per 10 years) | 1.03 (1.01 to 1.05) | 0.0066** | 1.19 (0.93 to 1.54) | 0.172 |
| Sex (male) | 0.95 (0.48 to 1.87) | 0.891 | — | — |
| Race (ref = White) | | | | |
| African American | 0.78 (0.32 to 1.92) | 0.582 | — | — |
| Asian | 0.50 (0.08 to 4.08) | 0.470 | — | — |
| Other | 0.74 (0.33 to 1.63) | 0.453 | — | — |
| Ethnicity (Hispanic) | 0.73 (0.37 to 1.44) | 0.360 | — | — |
| BMI (>30 obese) | 1.82 (0.82 to 4.08) | 0.141 | — | — |
| ACE inhibitor | 2.76 (1.13 to 7.83) | 0.0372* | 1.73 (0.57 to 6.06) | 0.354 |
| ARB | 0.61 (0.21 to 1.83) | 0.361 | 0.33 (0.09 to 1.19) | 0.0860 |
| Any Comorbidity | 2.12 (1.07 to 4.22) | 0.0310* | 1.55 (0.65 to 3.67) | 0.317 |
| Ventilated | | | | |
| Ang II (per 10 pg/mL) | 1.03 (1.00 to 1.06) | 0.0869 | 1.00 (1.00 to 1.01) | 0.163 |
| Ang 1-7 (per 10 pg/mL) | 0.97 (0.94 to 0.99) | 0.021* | 0.97 (0.94 to 0.99) | 0.0455* |
| Age (per 10 years) | 1.07 (0.89 to 1.28) | 0.484 | — | — |
| Sex (male) | 1.40 (0.74 to 2.68) | 0.302 | — | — |
| Race (ref = White) | | | | |
| African American | 0.66 (0.27 to 1.54) | 0.341 | — | — |
| Asian | 1.15 (0.14 to 7.47) | 0.882 | — | — |
| Other | 1.28 (0.62 to 2.68) | 0.503 | — | — |
| Ethnicity (Hispanic) | 1.29 (0.68 to 2.45) | 0.435 | — | — |
| BMI (>30 obese) | 1.47 (0.75 to 2.89) | 0.263 | — | — |
| ACE inhibitor | 1.78 (0.86 to 3.70) | 0.121 | — | — |
| Angiotensin receptor blocker | 0.43 (0.09 to 1.44) | 0.210 | — | — |
| Any Comorbidity | 0.89 (0.47 to 1.70) | 0.713 | — | — |
| Mortality | | | | |
| Ang II (per 10 pg/mL) | 1.05 (1.02 to 1.09) | 0.00413** | 1.04 (1.01 to 1.08) | 0.0302* |
| Ang 1-7 (per 10 pg/mL) | 0.96 (0.91 to 1.00) | 0.106 | 1.00 (0.99 to 1.00) | — |
| Age (per 10 years) | 1.73 (1.30 to 2.38) | 0.000319* | 1.65 (1.19 to 2.41) | 0.00497** |
| Sex (male) | 1.25 (0.53 to 3,05) | 0.618 | — | — |
| Race (ref = White) | | | | |
| African American | | | — | — |
| Asian | | | — | — |
| Other | | | — | — |
| Ethnicity (Hispanic) | 0.65 (0.25 to 1.57) | 0.358 | — | — |
| BMI (>30 obese) | 0.63 (0.25 to 1.57) | 0.318 | — | — |
| ACE inhibitor | 1.65 (0.61 to 4.21) | 0.307 | — | — |
| ARB | 1.57 (0.33 to 5.64) | 0.523 | — | — |
| Any Comorbidity | 2.96 (1.13 to 9.31) | 0.0398* | 1.31 (0.41 to 4.76) | 0.657 |

*OR, 95% CI and P-values obtained from univariable and multivariable regression models. Independent variables identified in univariable models to be associated with adverse outcomes (p < 0.10) were included in a multivariable binomial logistic regression.

TABLE 3
Patient Demographics and Characteristics by Ang 1-7 Quartile

| | Angiotensin 1-7 Quartile | | | | | | | | |
|---|---|---|---|---|---|---|---|---|---|
| | 1^{st} Quartile | | 2^{nd} Quartile | | 3^{rd} Quartile | | Top Quartile | | |
| | (n = 42) | | (n = 41) | | (n = 41) | | (n = 41) | | P value |
| Demographics | | | | | | | | | |
| Age (mean, SD) | 62.07 | (16.64) | 56.63 | (18.92) | 60.05 | (16.79) | 49.10 | (16.06) | 0.004 |
| Sex (%) | | | | | | | | | 0.471 |
| Female | 18 | (42.9) | 15 | (36.6) | 19 | (46.3) | 22 | (53.7) | |
| Male | 24 | (57.1) | 26 | (63.4) | 22 | (53.7) | 19 | (46.3) | |
| Race (%) | | | | | | | | | 0.055 |
| African American | 13 | (31.0) | 7 | (17.1) | 12 | (29.3) | 7 | (17.1) | |
| Asian | 1 | (2.4) | 0 | (0.0) | 0 | (0.0) | 4 | (9.8) | |
| Other | 12 | (28.6) | 14 | (34.1) | 16 | (39.0) | 19 | (46.3) | |
| White or Caucasian | 16 | (38.1) | 20 | (48.8) | 13 | (31.7) | 11 | (26.8) | |
| Ethnicity (%) | | | | | | | | | 0.606 |
| Hispanic | 15 | (35.7) | 17 | (41.5) | 15 | (36.6) | 20 | (48.8) | |
| Comorbidities & Medications | | | | | | | | | |
| Cardiac dysfunction | 12 | (28.6) | 7 | (17.1) | 4 | (9.8) | 4 | (9.8) | 0.066 |
| Chronic Kidney Disease | 7 | (16.7) | 7 | (17.1) | 6 | (14.6) | 8 | (19.5) | 0.95 |
| Lung Disease | 7 | (16.7) | 8 | (19.5) | 8 | (19.5) | 4 | (9.8) | 0.592 |
| Liver Disease | 0 | (0.0) | 1 | (2.4) | 2 | (4.9) | 0 | (0.0) | 0.287 |
| Stroke | 3 | (7.1) | 4 | (9.8) | 3 | (7.3) | 2 | (4.9) | 0.867 |
| Immunosuppression | 3 | (7.1) | 2 | (4.9) | 4 | (9.8) | 2 | (4.9) | 0.785 |
| Cancer | 4 | (9.5) | 4 | (9.8) | 4 | (9.8) | 2 | (4.9) | 0.822 |
| Diabetes | 19 | (45.2) | 17 | (41.5) | 14 | (34.1) | 17 | (41.5) | 0.775 |
| Body Mass Index | 15 | (38.5) | 13 | (37.1) | 18 | (51.4) | 16 | (47.1) | 0.562 |
| <30 (not obese) | 24 | (61.5) | 22 | (62.9) | 17 | (48.6) | 18 | (52.9) | |
| >30 (obese) | 12 | (28.6) | 7 | (17.1) | 4 | (9.8) | 4 | (9.8) | |
| ACE inhibitor prior to admission | 14 | (33.3) | 12 | (29.3) | 8 | (19.5) | 7 | (17.1) | 0.176 |
| ARB prior to admission | 4 | (9.5) | 6 | (14.6) | 4 | (9.8) | 1 | (2.4) | 0.176 |
| COVID-19 Clinical Indicators | | | | | | | | | |
| Symptom Onset to Sample (Days) | 11.65 | (8.70) | 14.21 | (17.73) | 10.55 | (8.06) | 5.74 | (4.32) | 0.024 |
| Mean Arterial Pressure | 86.28 | (11.13) | 87.59 | (12.95) | 90.31 | (10.69) | 93.39 | (13.23) | 0.047 |
| D-dimer³ | 1,042.64 | (897.64) | 603.33 | (282.71) | 1,001.50 | (1,553.44) | 422.67 | (479.44) | 0.315 |
| Hospitalization Status (%) | | | | | | | | | 0.002 |
| Admitted | 41 | (97.6) | 36 | (87.8) | 36 | (87.8) | 28 | (68.3) | |
| Not Admitted | 1 | (2.4) | 5 | (12.2) | 5 | (12.2) | 13 | (31.7) | |
| Oxygen Requirement (%) | | | | | | | | | 0.299 |
| None | 7 | (16.7) | 13 | (31.7) | 12 | (29.3) | 15 | (36.6) | |
| Low-flow oxygen | 17 | (40.5) | 11 | (26.8) | 12 | (29.3) | 14 | (34.1) | |
| High-flow oxygen | 0 | (0.0) | 1 | (2.4) | 0 | (0.0) | 2 | (4.9) | |
| Mechanical ventilation | 18 | (42.9) | 16 | (39.0) | 17 | (41.5) | 10 | (24.4) | |
| Mortality (%) | | | | | | | | | 0.29 |
| Deceased | 9 | (21.4) | 8 | (19.5) | 4 | (9.8) | 4 | (9.8) | |
| Recovered | 33 | (78.6) | 33 | (80.5) | 37 | (90.2) | 37 | (90.2) | |

TABLE 4
COVID-19 Patient Demographics and Clinical Characteristics by Outcome*

| | Number (%) | | | | | | | |
|---|---|---|---|---|---|---|---|---|
| | Hospitalized | | Required Oxygen | | Ventilated | | Deceased | |
| | (n = 142) | | (n = 118) | | (n = 61) | | (n = 25) | |
| Demographics | | | | | | | | |
| Age (mean, SD) | 59.34 | (16.72) | 59.49 | (16.04) | 58.33 | (14.27) | 69.48 | (15.74) |
| Female | 64 | (45.1) | 53 | (44.9) | 24 | (39.3) | 10 | (40.0) |
| Male | 78 | (54.9) | 65 | (55.1) | 37 | (60.7) | 15 | (60.0) |
| African American | 32 | (22.5) | 28 | (23.7) | 11 | (18.0) | 5 | (20.0) |
| Asian | 3 | (2.1) | 3 | (2.5) | 2 | (3.3) | 0 | (0.0) |
| Other | 51 | (35.9) | 42 | (35.6) | 26 | (42.6) | 6 | (24.0) |
| White or Caucasian | 56 | (39.4) | 45 | (38.1) | 22 | (36.1) | 14 | (56.0) |
| Hispanic | 57 | (40.1) | 45 | (38.1) | 27 | (44.3) | 8 | (32.0) |
| Comorbidities | | | | | | | | |
| Cardiac dysfunction | 27 | (19.0) | 21 | (17.8) | 8 | (13.1) | 8 | (32.0) |
| Chronic Kidney Disease | 28 | (19.7) | 22 | (18.6) | 6 | (9.8) | 8 | (32.0) |
| Lung Disease | 26 | (18.3) | 20 | (16.9) | 7 | (11.5) | 5 | (20.0) |
| Liver Disease | 3 | (2.1) | 2 | (1.7) | 0 | (0.0) | 0 | (0.0) |
| Stroke | 12 | (8.5) | 11 | (9.3) | 3 | (4.9) | 5 | (20.00) |
| Immunosuppression | 11 | (7.7) | 9 | (7.6) | 7 | (11.5) | 2 | (8.0) |
| Cancer | 13 | (9.2) | 10 | (8.5) | 6 | (9.8) | 3 | (12.0) |
| Diabetes | 62 | (43.7) | 54 | (45.8) | 24 | (39.3) | 13 | (52.0) |
| Body Mass Index | | | | | | | | |
| <30 (not obese) | 56 | (43.1) | 45 | (40.5) | 23 | (37.7) | 12 | (54.5) |
| >30 (obese) | 74 | (56.9) | 66 | (59.5) | 38 | (62.3) | 10 | (45.5) |
| Medications | | | | | | | | |
| ACE inhibitor | 39 | (27.5) | 35 | (29.7) | 20 | (32.8) | 8 | (32.0) |
| Angiotensin receptor blocker | 14 | (9.9) | 9 | (7.6) | 3 | (4.9) | 3 | (12.0) |
| COVID-19 Clinical Indicators/Treatments | | | | | | | | |
| Received Steroids (%)¹ | 72 | (50.7) | 67 | (56.8) | 41 | (62.1) | 12 | (48.0) |
| Received Remdesivir (%)² | 38 | (27.1) | 39 | (33.3) | 17 | (27.9) | 5 | (20.8) |
| D-dimer³ (Mean, SD) | 795.06 | (1006.82) | 776.94 | (1030.79) | 1043.61 | (1286.46) | 897.00 | (838.89) |
| MAP (Day of Sample) (Mean, SD) | 88.01 | (11.67) | 87.07 | (11.32) | 83.59 | (10.16) | 82.96 | (11.25) |
| Days from Symptom Onset to Sample | 11.57 | (11.57) | 11.35 | (10.44) | | | | |
| (Mean, SD) | | | | | | | | |

*Patient demographics and clinical characteristics by Angiotensin 1-7 quartile. P-values obtained from chi-square analysis.

1. A method for treating a subject with a coronavirus infection, the method comprising:
(a) providing a subject infected with a coronavirus resulting in and/or having a prothrombotic condition in addition to being infected by a coronavirus; and
(b) administering to the subject an angiotensin (1-7) peptide or an analog or derivative thereof, a Mas Receptor (MasR) agonist, or any combination thereof.

2. The method of claim 1, wherein the coronavirus is a SARS-CoV-1, MERS, or SARS-CoV-2 coronavirus.

3. The method of claim 1, wherein the subject is suffering from COVID-19 disease.

4. The method of claim 1, wherein the subject has and/or is at risk for developing a thrombotic complication, optionally a thrombotic complication selected from the group consisting of acute limb ischemia, abdominal and/or thoracic aortic thrombosis, mesenteric ischemia, myocardial infarction, venous thromboembolism, pulmonary embolism, cerebrovascular accident, and any form of systemic arterial embolism.

5. The method of claim 1, wherein the subject is at an elevated risk of an adverse pregnancy outcome, optionally wherein the adverse pregnancy outcome is selected from the group consisting of intrauterine fetal death, stillbirth, fetal growth restriction, and preterm birth secondary to placental insufficiency, and further wherein the adverse pregnancy outcome results from and/or is secondary to thrombosis formation in the subject associated with the coronavirus infection.

6. The method of claim 1, wherein the subject is at risk of a complication resulting from the coronavirus infection due to an underlying prothrombotic state, optionally wherein the underlying prothrombotic state results from pregnancy, malignancy, a genetic condition, and/or a rheumatologic condition that predisposes the subject to thrombosis formation.

7. The method of claim 1, wherein the administration is parenteral, rectal, oral, or a combination thereof.

8. The method of claim 7, wherein the parenteral administration is intravenous, subcutaneous, inhalation, intradermal, transdermal, and/or transmucosal administration.

9. The method of claim 1, wherein the angiotensin (1-7) peptide comprises an amino acid sequence selected from the group consisting of Asp-Arg-Val-Tyr-Ile-His-Pro (SEQ ID NO: 1) or a functional equivalent thereof, Asp-Arg-Val-Ser-Ile-His-Cys (SEQ ID NO: 2) or a functional equivalent thereof, and Ala-Arg-Val-Ser-Ile-His-Cys (SEQ ID NO: 3) or a functional equivalent thereof.

10. The method of claim 1, wherein the angiotensin (1-7) peptide or the analog or derivative thereof is provided in composition comprising a degradable polymer having an electrostatic interaction with the angiotensin (1-7) peptide or the analog or derivative thereof, a non-degradable polymer having an electrostatic interaction with the angiotensin (1-7) peptide or the analog or derivative thereof, a non-degradable polymer having a hydrophobic interaction with the angiotensin (1-7) peptide or the analog or derivative thereof, a non-degradable polymer having a hydrogen bonding interaction with the angiotensin (1-7) peptide or the analog or derivative thereof, or any combination thereof.

11. The method of claim 1, wherein the Mas Receptor (MasR) agonist is N-(Ethylcarbamoyl)-3-(4-((5-formyl-4-methoxy-2-phenyl-1H-imidazol-1-YL)methyl)phenyl)-5-isobutylthiophene-2-sulfonamide (AVE0991), an analog thereof, a derivative thereof, or any combination thereof.

12. The method of claim 1, wherein the subject is suffering from weight loss and/or olfactory nerve invasion by the coronavirus.

13. The method of claim 1, further comprising administering to the subject an ACE inhibitor, an Angiotensin II Receptor Blocker (ARB), or any combination thereof.

14. The method of claim 13, wherein:
(i) the ACE inhibitor is selected from the group consisting of benazepril, captopril, enalapril, fosinopril, lisinopril, moexioril, perindopril, quinapril, ramipril, and trandolapril; and/or
(ii) the ARB is selected from the group consisting of candesartan, irbesartan, olmesartan, losartan, valsartan, telmisartan, and eprosartan;
or any combination thereof.

15. A composition comprising a degradable polymer having an electrostatic interaction with an angiotensin (1-7) peptide or the analog or derivative thereof, a non-degradable polymer having an electrostatic interaction with an angiotensin (1-7) peptide or the analog or derivative thereof, a non-degradable polymer having a hydrophobic interaction with an angiotensin (1-7) peptide or the analog or derivative thereof, a non-degradable polymer having a hydrogen bonding interaction with an angiotensin (1-7) peptide or the analog or derivative thereof, or any combination thereof.

16. The composition of claim 15, wherein the composition is formulated for the treatment of a subject infected with a coronavirus and having a prothrombic condition in addition to being infected by a coronavirus.

17. The composition of claim 16, wherein the subject is suffering from weight loss and/or olfactory nerve invasion by the coronavirus.

18. Use of an angiotensin (1-7) peptide and/or an analog or derivative thereof and/or a Mas Receptor (MasR) agonist for the preparation for medicament for treating a subject infected with a coronavirus, wherein the subject is a subject infected with a coronavirus and having a prothrombic condition in addition to being infected by a coronavirus.

19. Use of an angiotensin (1-7) peptide and/or an analog or derivative thereof and/or Mas Receptor (MasR) agonist for treating a subject infected with a coronavirus, wherein the subject is a subject infected with a coronavirus and having a prothrombic condition in addition to being infected by a coronavirus.

20. Use according to claim 18, wherein the angiotensin (1-7) peptide and/or the analog or derivative thereof is provided in a composition comprising a degradable polymer having an electrostatic interaction with the angiotensin (1-7) peptide and/or the analog or derivative thereof, a non degradable polymer having an electrostatic interaction with the angiotensin (1-7) peptide and/or the analog or derivative thereof, a non-degradable polymer having a hydrophobic interaction with the angiotensin (1-7) peptide and/or the analog or derivative thereof, a non-degradable polymer having a hydrogen bonding interaction with the angiotensin (1-7) peptide and/or the analog or derivative thereof, or any combination thereof.

21. Use according to claim 18, wherein the subject is suffering from weight loss and/or olfactory nerve invasion by the coronavirus.


SEQUENCE LISTING (16 sequences)

SEQ ID NO: 1 - protein, 7 aa, Artificial Sequence
  Artificially synthesized Ang 1-7 peptide sequence
DRVYIHP

SEQ ID NO: 2 - protein, 7 aa, Artificial Sequence
  Artificially synthesized Ang 1-7 peptide sequence
DRVSIHC

SEQ ID NO: 3 - protein, 7 aa, Artificial Sequence
  Artificially synthesized Ang 1-7 peptide sequence
ARVSIHC

SEQ ID NO: 4 - DNA, 24 bp, Artificial Sequence
  Artificially synthesized oligonucleotide sequence
  misc_feature (1)..(1): The nucleotide at the 5′ end is optionally labeled with a FAM moiety
  misc_feature (2)..(2): y = t/u or c
  misc_feature (24)..(24): The nucleotide at the 3′ end is optionally labeled with a BHQ1 moiety
aycacattggcacccgcaatcctg

SEQ ID NO: 5 - DNA, 3596 bp, Homo sapiens
  CDS (307)..(2724)
ggcactcatacatacactctggcaatgaggacactgagctcgcttctgaaatttgacaagataaccactaaaatctctttgaattctatgttgttgtgatcccatggctacagaggatcaggagttgacatagatactctttggatttcataccatgtggaggctttcttacttccacgtgaccttgactgagttttgaatagcgcccaacccaagttcaaaggctgataagagagaaaatctcatgaggaggttttagtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 6 - protein, 805 aa, Homo sapiens [sequence omitted]

SEQ ID NO: 7 - DNA, 3339 bp, Homo sapiens
  CDS (50)..(2467)
agtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 8 - protein, 805 aa, Homo sapiens [sequence omitted]

SEQ ID NO: 9 - DNA, 3141 bp, Homo sapiens
  CDS (50)..(2410)
agtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagccaactccactcttgggaaaaagttggctgacagccatcttgaaagattgagggctgaaaatccaagaactgaggatcaagatctctcccctgtcataaaactacatatggatctgcccttcagtaggaaattcctaaaagtctcccatgagataaagaatcagtgctggaaaactcactccgataccaccaccaccaaatcatgatagaaacagctatgtgtgtctttttttaattagacctcatcttccttggaactaactctgaaagggccatgaatctcagcccccccaaaatccctccccaaaagcatgctgccaggtgatgcaggcccaagctaggtgacagatgtttaacttggaatgatgtttgcagtcatgtgataataacattggatggaacaattcagaggctgttcttatgattacaagtaatggggacatttttatcatttgagaatgactgcaaaactatggaatttggcaaagactttatttggaagcagggaagaaagcccactgaatagctttgaagggataatggagggaaagaattatgttgttttctgcttttgtcctatagagtttcatttcaacaccaggatacttccacaaagcagtcttggccatgttgatggtaaggaaagaatgacagctaataacagctgcctgttatgtgtgatgccatcttaaggacatctcccgcatgcacccattttttctttttttttttttggtgactatttatgggcttactggctaggaaaagacacaacaatgaaa

SEQ ID NO: 10 - protein, 786 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKPTPLLGKSWLTAILKD

SEQ ID NO: 11 - DNA, 3006 bp, Homo sapiens
  CDS (50)..(2134)
agtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 12 - protein, 694 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLLEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 13 - DNA, 2360 bp, Homo sapiens
  CDS (109)..(1488)
gtaattcccaggttgcaggcttgtgagagccttaggttggattccctagcttgaaaaggagatcgttttacaagtgcttcattgaggagagctctgaggcagaggggaatgagggaagcaggctgggacaaaggagggaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 14 - protein, 459 aa, Homo sapiens
MREAGWDKGGRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 15 - DNA, 3135 bp, Homo sapiens
  CDS (179)..(2263)
ttagaactttttaaaagaggcaaaggcagaggagaacaaaggaaggaggaagtaacttgtggaatgttgagaaagcgcccaacccaagttcaaaggctgataagagagaaaatctcatgaggaggttttagtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 16 - protein, 694 aa, Homo sapiens; identical to SEQ ID NO: 12